(19) United States
(12) Patent Application Publication
(10) Pub. No.: US 2001/0049102 A1
(43) Pub. Date: Dec. 6, 2001

(54) Methods for determining single nucleotide variations

(76) Inventors: Xiaohua C. Huang, Mountain View, CA (US); Michael James Gilchrist, Cambridge (GB); L. Gordon Bentley, Alameda, CA (US); Hugh Y. Rienhoff JR., San Carlos, CA (US)

Correspondence Address: TOWNSEND AND TOWNSEND AND CREW, TWO EMBARCADERO CENTER, EIGHTH FLOOR, SAN FRANCISCO, CA, 94111-3834, US

(21) Appl. No.: 09/792,413

(22) Filed: Feb. 23, 2001

Related U.S. Application Data
(60) Provisional application No. 60/184,808, filed on Feb. 24, 2000.

(51) Int. Cl. C12Q 1/68

(52) U.S. Cl. 435/6

(57) ABSTRACT

The present invention provides a variety of methods for determining the identity of a nucleotide present at a variant site in a target nucleic acid. The methods involve conducting partial chain termination sequencing reactions of a target nucleic acid of interest conducted with the four deoxynucleotides but only one to three non-extendible nucleotides (i.e., analogs of dATP, dTTP, dGTP and dCTP that are non-extendible by a polymerase). The non-extendible nucleotide(s) utilized in the sequencing reactions are selected to be complementary to the bases potentially occupying the variant site of a target nucleic acid that serves as a template during sequencing. Analyses can also be performed in multiplex formats.

### CROSS-REFERENCE TO RELATED APPLICATIONS

This application claims the benefit of U.S. Provisional Application No. 60/184,808, filed Feb. 24, 2000, which is incorporated herein by reference in its entirety for all purposes.

### FIELD OF THE INVENTION

The present invention relates to the field of molecular genetics, particularly the identification and detection of certain nucleotide sequences.

### BACKGROUND OF THE INVENTION

The nucleic acids comprising the genome of an organism contain the genetic information for that organism. The translation or expression of these nucleic acids generates proteins that function in many diverse ways within the organism. Even minute changes in a nucleotide sequence, including single base pair substitutions, can have a significant effect in the quality or quantity of a protein. Single nucleotide changes are referred to as single nucleotide polymorphisms or simply SNPs, and the site at which the SNP occurs is typically referred to as a polymorphic site.

Many SNPs, as well as larger nucleic acid alterations, can affect the phenotype of the organism, and in some instances can result in the onset of disease. For example, diseases associated with SNPs include: sickle cell anemia, β-thalassemias, diabetes, cystic fibrosis, hyperlipoproteinemia, a wide variety of autoimmune diseases, and the formation of oncogenes. In addition to causing or affecting disease states, point mutations can cause altered pathogenicity and resistance to therapeutics that target certain microorganisms.

The ability to detect specific nucleotide alterations or mutations in DNA sequences has a number of medical and non-medical utilities. For example, methods capable of identifying nucleotide alterations provide a means for screening and diagnosing many common diseases that are associated with SNPs. Methods that can quickly identify such changes or mutations are also valuable in taking prophylactic measures, assessing the propensity for disease, and in patient counseling and education. As for non-medical applications, such methods have value in the detection of microorganisms, resolving paternity disputes and in forensic analysis to identify perpetrators of crimes.

Various methods have been developed to obtain sequence information for variant sites that include nucleotide alternations or mutations such as SNPs. These methods include: (i) sequencing methods, hybridization reactions between a target nucleic acid and allele-specific oligonucleotide (ASO) probes (see, e.g., European Patent Publications EP-237362 and EP-32931 1), (ii) allele specific amplification (see, e.g., U.S. Pat. Nos. 5,521,301; 5,639,611; and 5,981,176), (iii) quantitative RT-PCR methods [e.g., the so-called “TaqMan assays”; see, e.g., U.S. Pat Nos. 5,210,015 to Gelfand, 5,538,848 to Livak, et al., and 5,863,736 to Haaland, as well as Heid, C. A., et al., Genome Research, 6:986-994 (1996); Gibson, U. E. M, et al., Genome Research 6:995-1001 (1996); Holland, P. M., et al, Proc. Natl Acad. Sci. USA 88:7276-7280, (1991); and Livak, K. J., et al., PCR Methods and Applications 357-362 (1995)], and (iv) various single base pair extension (SBPE) assays.

Two widely used sequencing methods include the chain-terminator method developed by Sanger et al. [see, e.g., Proc. Nat'l Acad. Sci. USA 74:5463-5467 (1977)] and the chain degradation sequencing method developed by Maxam and Gilbert [see, e.g., Proc. Nat'l. Acad. Sci. USA 74:560-564 (1977)]. Although the complete sequence information that can be obtained by these methods is useful for some applications, such complete sequence data is unnecessary if one is only interested in determining the identity of a single nucleotide at a variant site in a nucleic acid of interest. Hence, the use of these methods for such determinations typically is not justified from a time and cost basis.

A number of SPBE assays have been developed, but the general approach is quite similar. Typically, these assays involve hybridizing a primer that is complementary to a target nucleic acid such that the 3′ end of the primer is immediately 5′ of the variant site or is adjacent thereto. Hybridization is conducted in the presence of a polymerase and one or more labeled non-extendible nucleotides that are complementary to the nucleotide(s) that potentially occupy the variant site. The non-extendible nucleotide is a nucleotide analog that prevents further extension by the polymerase once incorporated into the primer. If the added non-extendible nucleotide(s) is(are) complementary to the nucleotide at the variant site, then a labeled non-extendible nucleotide is incorporated onto the 3′ end of the primer to generate a labeled extension product. Hence, extended primers provide an indication of which nucleotide is present at the variant site of a target nucleic acid. Such methods are discussed, for example, in U.S. Pat. Nos. 5,846,710; 6,004,744; 5,888,819; 5,856,092; 5,710,028 and in PCT publication WO 92/16657.

SBPE methods also suffer from certain shortcomings. One potential problem is that the primer binds to the wrong site on the target nucleic acid. This problem can arise for a variety of reasons. For example, the region to which the primer is to bind in some instances can have secondary structure that impedes hybridization and results in mispriming. Segments that have sequences similar to those of the intended primer binding site can also result in mispriming or “cross talk.” Such mispriming results in the generation of unintended extension product that can result in the miscalling of the base at the variant site of interest. SPBE methods also generally require that different primers be designed for each variant site that one wishes to interrogate. The need to tailor the primers makes the methods somewhat tedious, time consuming to perform and limits the flexibility of the methods. Further, the methods often require labeled non-extendible nucleotides which can be quite expensive.

### SUMMARY

A variety of methods are disclosed herein for determining which of a plurality of potential nucleotides are present at one or more sites of one or more target nucleic acids. Thus, the methods are useful for determining the identity of a nucleotide at a variant site of a target nucleic acid, such as a polymorphic site or a site having a mutation. In general the methods involve conducting partial chain termination sequencing reactions, with reactions being conducted in the presence of the four deoxynucleotides (i.e., dATP, dTTP, dCTP and dGTP) but only one to three non-extendible nucleotides. The particular non-extendible nucleotides utilized in the analysis are selected to be complementary to one of the bases potentially located at the variant site of the target nucleic acid that functions as the template strand during the partial sequencing reactions. The methods can be utilized in any of a variety of applications in which it is useful to know the identity of a nucleotide at a site of variation in a nucleic acid of interest. Thus, the methods are useful in a variety of clinical applications (e.g., in detecting the presence of particular polymorphic forms), in genotyping and in the identification of individuals (e.g., forensic applications).

Certain of the methods involve partially sequencing the target nucleic acid by conducting template-dependent primer extension reactions. The target nucleic acid serves as a template, and the reactions are conducted in the presence of the four deoxynucleotides (dATP, dTTP, dCTP and dGTP) and one to three non-extendible nucleotides, each non-extendible nucleotide selected to be complementary to a different base potentially occupying the variant site of the target nucleic acid, whereby a plurality of extension products of differing size are formed. The extension products formed include: (i) a first extension product, if a first of the non-extendible nucleotides is present and complementary to the base occupying the variant site of the target nucleic acid, (ii) a second extension product, if a second of the non-extendible nucleotides is present and complementary to the base occupying the variant site of the target nucleic acid, and (iii) a third extension product, if a third of the non-extendible nucleotides is present and complementary to the base occupying the variant site of the target nucleic acid. Because the first, second and third extension products are only formed when a particular base is present in the target nucleic acid, they are characteristic products whose presence indicates which nucleotide(s) is/are present in the target nucleic acid(s). Hence, following formation of the extension products, the presence or absence of the first, second and/or third extension product(s) are detected as an indication of the base occupying the variant site of the target nucleic acid.

Various options are available to facilitate detection of the first, second and/or third extension products. One option is to conduct the extension reactions with labeled primers such that the resulting extension products, including the first, second and third extension products, if formed, are labeled. Another option is to label the extension products after they have been generated. This can be accomplished using an intercalation dye, for example. Yet another option is to utilize labeled non-extendible nucleotides. By using differentially labeled non-extendible nucleotides, extension reactions, even if conducted with two or three non-extendible nucleotides, can be conducted in a single reaction vessel, as the particular non-extendible nucleotide incorporated into the primer can be determined on the basis of the identity of the incorporated label.

In many instances, the various extension products are separated according to size before the extension products are detected. Often this is done on a single gel electrophoresis lane or within a single capillary. A comparison of signal magnitude for one of the characteristic products (i.e., a product formed if a particular nucleotide occupies the variant site of the target nucleic acid) with that of one or more other extension products can be useful in a number of different ways. For example, the relative signal magnitudes for a characteristic extension product relative to the signal for another product can indicate whether a sample from a diploid subject is homozygous or heterozygous. Such comparisons can be made in a straightforward manner when the extension products are size-separated via gel electrophoresis to obtain an electropherogram (a plot in which signal intensity for an extension product is recorded as a function of extension product size).

Thus, for instance, one common analysis involves the determination of the genotype of a diploid subject for a biallelic polymorphic site in which either a first or second nucleotide (e.g., A or G) is present at the polymorphic site. In one form, the analysis involves conducting partial sequencing reactions in the presence of a single non-extendible nucleotide (plus the four deoxynucleoside triphosphates), whereby a series of differing size extension products are generated depending upon when during the extension reaction the non-extendible nucleotide is incorporated onto the 3′ end of the primer. Characteristic extension product is formed only if the non-extendible nucleotide is complementary to the nucleotide(s) occupying the variant site of one or both copies of the target nucleic acid in the sample obtained from the diploid subject. The signals for the other extension products, however, tend to correlate with extension products formed for sites in which both copies of the target nucleic acid include a complementary nucleotide; hence, the magnitude of these signals represents a homozygous condition. Thus, the absence of a signal for the characteristic product, or the presence of a signal that has roughly the same magnitude as that for other extension products (e.g., signals adjacent the characteristic signal in an electropherogram), indicates that the subject is homozygous; the identity of the nucleotide occupying the variant site can be deduced from the identity of the non-extendible nucleotide utilized in the analysis. If, however, the magnitude of the characteristic signal is only roughly half that of the signal for other extension products, then the subject is a heterozygote.

As indicted supra, analyses can be conducted with only one, two or three non-extendible nucleotides (plus the four deoxynucleotides). Analyses conducted with a single non-extendible nucleotide typically involve partially sequencing a target nucleic acid by conducting template-dependent extension reactions with the target nucleic acid serving as template in the presence of the four deoxynucleotides (dATP, dTTP, dCTP and dGTP) and a single non-extendible nucleotide selected to be complementary to a base potentially occupying the variant site of the template nucleic acid. The result is the formation of a plurality of extension products of differing size, with the extension products including, in part, a first extension product, provided the single non-extendible nucleotide is complementary to the base occupying the variant site of the target nucleic acid. The presence or absence of the first extension product is then detected as an indication of the base occupying the variant site of the target nucleic acid.

Analyses with two non-extendible nucleotides generally involve partially sequencing the target nucleic acid by conducting template-dependent primer extension reactions with the target nucleic acid serving as template in the presence of the four deoxynucleotides (dATP, dTTP, dCTP and dGTP) and a first and a second non-extendible nucleotide, the non-extendible nucleotides selected to be complementary with the bases potentially occupying the variant site of the target nucleic acid. As a consequence of the random incorporation of the non-extendible nucleotide at different points during the extension reaction, a plurality of extension products of differing size are generated, some products formed by incorporation of the first non-extendible nucleotide onto the 3′ end of the primer and others formed by incorporation of the second non-extendible nucleotide at the 3′ end of the primer. The extension products include a first extension product if the first non-extendible nucleotide is complementary to the base occupying the variant site of the target nucleic acid, and a second extension product if the second non-extendible nucleotide is complementary to the base occupying the variant site of the target nucleic acid. The presence or absence of the first and second extension product is detected to determine which base occupies the variant site of the target nucleic acid.

Analyses with three non-extendible nucleotides typically involve partially sequencing the target nucleic acid by conducting template-dependent primer extension reactions with the target nucleic acid serving as template in the presence of four deoxynucleotides (dATP, dTTP, dCTP and dGTP) and a first, a second and a third non-extendible nucleotide, the non-extendible nucleotides selected to be complementary with the bases potentially occupying the variant site of the target nucleic acid, whereby a plurality of extension products of differing size are generated. The extension products formed include, in part, (i) a first extension product if the first non-extendible nucleotide is complementary to the base occupying the variant site of the target nucleic acid, (ii) a second extension product if the second non-extendible nucleotide is complementary to the base occupying the variant site of the target nucleic acid, and (iii) a third extension product if the third non-extendible nucleotide is complementary to the base occupying the variant site of the target nucleic acid. The characteristic extension products (i.e., the first, second and third extension products) are detected, to obtain an indication of the base occupying the variant site of the target nucleic acid.

The analyses with two or three non-extendible nucleotides can be performed by conducting the extension reactions in a single reaction vessel, in which case the non-extendible nucleotides are typically differentially labeled. Alternatively, the target nucleic acid can be split between reaction vessels. A separate extension reaction is then conducted in each vessel in the presence of all four of the deoxynucleotides and one of the non-extendible nucleotides. By using differentially labeled primers in the different reaction vessels, the extension products can be pooled prior to detection. Which non-extendible nucleotide has been incorporated into any given extension product can be determined from the identity of the label borne by the extended primer.

### BRIEF DESCRIPTION OF THE DRAWINGS

FIG. 1 illustrates the major steps in certain methods of the invention utilizing partial sequencing to identify the nucleotide occupying the variant site on a nucleic acid of interest.

FIG. 2 illustrates an example of a partial sequencing method utilizing a single nucleotide.

FIG. 3 shows an example of certain steps in a partial sequencing method using two nucleotides.

FIG. 4 depicts certain steps in a genotyping method in which two nucleotides are used to conduct partial sequencing reactions.

FIG. 5 illustrates a multiplexing method of the invention for samples obtained from two subjects.

FIGS. 6A-C depict electropherograms obtained during a genotyping analysis of a single nucleotide polymorphism for different subjects using a two nucleotide partial sequencing method of the invention.

FIGS. 7A-I depict electropherograms obtained during a genotyping analysis of a single nucleotide polymorphism for different subjects using a two nucleotide and a single nucleotide partial sequencing method of the invention.

### DETAILED DESCRIPTION

### I. Definitions

A “nucleic acid” is a deoxyribonucleotide or ribonucleotide polymer in either single or double-stranded form, including known analogs of natural nucleotides unless otherwise indicated.

A “polynucleotide” refers to a single- or double-stranded polymer of deoxyribonucleotide or ribonucleotide bases.

An “oligonucleotide” is a single-stranded nucleic acid typically ranging in length from 2 to about 500 bases. Oligonucleotides are often synthetic but can also be produced from naturally occurring polynucleotides. Oligonucleotides can be prepared by any suitable method, including, for example, cloning and restriction of appropriate sequences and direct chemical synthesis by a method such as the phosphotriester method of Narang et al., Meth. Enzymol. 68:90-99 (1979); the phosphodiester method of Brown et al., Meth. Enzymol. 68:109-151 (1979); the diethylphosphoramidite method of Beaucage et al., Tetrahedron Lett. 22:1859-1862 (1981); and the solid support method described in U.S. Pat. No. 4,458,066.

A “primer” is a single-stranded oligonucleotide capable of acting as a point of initiation of template-directed DNA synthesis under appropriate conditions (e.g., in the presence of four different nucleoside triphosphates and an agent for polymerization, such as, DNA or RNA polymerase or reverse transcriptase) in an appropriate buffer and at a suitable temperature. The appropriate length of a primer depends on the intended use of the primer but typically ranges from 15 to 30 nucleotides. Short primer molecules generally require cooler temperatures to form sufficiently stable hybrid complexes with the template. A primer need not reflect the exact sequence of the template but must be sufficiently complementary to hybridize with a template. The term “primer site” refers to the area of the target DNA to which a primer hybridizes. The term “primer pair” means a set of primers including a 5′ “upstream primer” that hybridizes with the 5′ end of the DNA sequence to be amplified and a 3′ “downstream primer” that hybridizes with the complement of the 3′ end of the sequence to be amplified.

A primer that is “perfectly complementary” has a sequence fully complementary across the entire length of the primer and has no mismatches. The primer is typically perfectly complementary to a portion (subsequence) of a target sequence. A “mismatch” refers to a site at which the nucleotide in the primer and the nucleotide in the target nucleic acid with which it is aligned are not complementary.

The term “substantially complementary” means that a primer is not perfectly complementary to its target sequence; instead, the primer is only sufficiently complementary to hybridize selectively to its respective strand at the desired primer binding site.

The phrase “hybridizing specifically to”, refers to the binding, duplexing, or hybridizing of a molecule only to a particular nucleotide sequence under stringent conditions when that sequence is present in a complex mixture (e.g., total cellular) DNA or RNA.

The term “stringent conditions” refers to conditions under which a primer will hybridize to its target subsequence, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. In other instances, stringent conditions are chosen to be about 20° C. or 25° C. below the melting temperature of the sequence and a probe with exact or nearly exact complementarity to the target. As used herein, the melting temperature is the temperature at which a population of double-stranded nucleic acid molecules becomes half-dissociated into single strands. Methods for calculating the Tₘ of nucleic acids are well known in the art (see, e.g., Berger and Kimmel (1987) Methods in Enzymology, vol. 152: Guide to Molecular Cloning Techniques, San Diego: Academic Press, Inc. and Sambrook et al. (1989) Molecular Cloning: A Laboratory Manual, 2nd ed., vols. 1-3, Cold Spring Harbor Laboratory), both incorporated herein by reference. As indicated by standard references, a simple estimate of the Tₘ value can be calculated by the equation: Tₘ=81.5+0.41(% G+C), when a nucleic acid is in aqueous solution at 1 M NaCl (see e.g., Anderson and Young, “Quantitative Filter Hybridization,” in Nucleic Acid Hybridization (1985)). Other references include more sophisticated computations which take structural as well as sequence characteristics into account for the calculation of Tₘ. The melting temperature of a hybrid (and thus the conditions for stringent hybridization) is affected by various factors such as the length and nature (DNA, RNA, base composition) of the probe and nature of the target (DNA, RNA, base composition, present in solution or immobilized, and the like), and the concentration of salts and other components (e.g., the presence or absence of formamide, dextran sulfate, polyethylene glycol). The effects of these factors are well known and are discussed in standard references in the art, see e.g., Sambrook, supra, and Ausubel, supra. Typically, stringent conditions will be those in which the salt concentration is less than about 1.0 M Na ion, typically about 0.01 to 1.0 M Na ion concentration (or other salts) at pH 7.0 to 8.3 and the temperature is at least about 30° C. for short probes or primers (e.g., 10 to 50 nucleotides) and at least about 60° C. for long probes or primers (e.g., greater than 50 nucleotides). Stringent conditions can also be achieved with the addition of destabilizing agents such as formamide.

A “site of variation” or “variant site” when used with reference to a nucleic acid broadly refers to a site wherein the identity of nucleotide at the site varies between nucleic acids that otherwise have similar sequences. For double-stranded nucleic acids, the variant site includes the variable nucleotide on one strand and the complementary nucleotide on the other strand. Thus, for template-dependent extension reactions, there is a variant site on the template strand and in the extension product once a nucleotide complementary to the nucleotide at the variant site of the template strand is incorporated into the extension product. A variant site can be the site of a single nucleotide polymorphism or the site of a somatic mutation, for example.

A “polymorphic marker” or “polymorphic site” is the locus at which divergence occurs. Preferred markers have at least two alleles, each occurring at frequency of greater than 1%, and more preferably greater than 10% or 20% of a selected population. The first identified allelic form is arbitrarily designated as the reference form and other allelic forms are designated as alternative or variant alleles. The allelic form occurring most frequently in a selected population is sometimes referred to as the wild-type form, whereas allelic forms occurring less frequently are referred to as mutant alleles. Diploid organisms may be homozygous or heterozygous for allelic forms. A diallelic polymorphism has two forms, a triallelic polymorphism has three forms and a tetraallelic polymorphism has four forms.

A “single nucleotide polymorphism” (SNP) occurs at a polymorphic site occupied by a single nucleotide, which is the site of variation between allelic sequences. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele.

The term “naturally occurring” as applied to an object means that the object can be found in nature.

The term “subject” and “individual” are used interchangeably herein to refer to any type of organism, but most typically is used to refer to a human.

### II. Overview

The present invention provides a variety of methods for determining the identity of a nucleotide present at a variant site in a target nucleic acid. The methods involve conducting partial chain termination sequencing reactions of a target nucleic acid of interest conducted with only one to three non-extendible nucleotides (i.e., analogs of dATP, dTTP, dGTP and dCTP that are non-extendible by a polymerase). The non-extendible nucleotide(s) utilized in the sequencing reactions are selected to be complementary to the bases potentially occupying the variant site of a target nucleic acid that serves as a template during sequencing (i.e., during primer extension reactions). It has unexpectedly been found that the sequencing products formed in sequencing reactions conducted with as few as a single nucleotide can be used to deduce the identity of the nucleotide at a variant site and to determine the genotype of diploid organisms.

Unlike the single base pair extension reactions discussed in the Background section, the chain termination sequencing reactions of the invention generate a plurality of differing sized extension products rather than only extension products extended by a single base. The differing sized fragments result from the random termination of the primer extension reactions occasioned by the incorporation of the non-extendible nucleotide(s) during the primer extension reactions. If the non-extendible nucleotide(s) utilized in the sequencing reactions is present at the variant site of the target nucleic acid acting as a template, then one of the plurality of extension products formed is a characteristic extension product. The characteristic extension product results from the incorporation of the non-extendible nucleotide into the variant site of the extension product. Thus, detection of the various characteristic products formed is an indication of the nucleotide present at the variant site of the target nucleic acid.

In certain methods, fragments of similar size to the characteristic extension product can serve a useful role. These similarly sized fragments correspond to extension products formed through incorporation of non-extendible nucleotides at sites adjacent to the variant site. If such fragments are separated by size and signals for the fragments obtained (e.g., as a trace or electropherogram), a fingerprint region characteristic of fragments neighboring the characteristic extension product can be obtained. By comparing the magnitude of the signal for the characteristic extension product with that of its neighbors, the identity of the nucleotide at the variant site and the genotype of a diploid organism can be readily deduced. The fingerprint region also aids computerized analysis of the extension products formed, as the fingerprint region can be used to rapidly pinpoint which peak in a trace corresponds to the variant site.

By limiting the number of sequencing reactions conducted, analyses can be conducted at lower cost. Because the methods are based on sequencing reactions, typically using target nucleic acids that have been tailored to include a primer binding sequence (e.g., the universal primer sequence), the methods are less susceptible to mispriming and the attendant inaccuracies in determining the identity of the nucleotide at the variant site. The methods can easily be adapted to conduct multiplex assays in which the identity of a nucleotide occupying a variant site in different target nucleic acids (e.g., target nucleic acids from different subjects) is determined in a single analysis.

The methods can be used in a number of different applications. For example, in the medical field, the methods of the invention can be used to determine which allele is present at a single nucleotide polymorphic (SNP) site or to detect mutations at a particular site. Because many diseases are associated with SNPs or mutations, the methods can be used in a variety of diagnostic, research and prognostic applications. In addition, for diploid subjects, the methods can be used to determine if the individual is homozygous or heterozygous for a particular allele at the variant site, i.e., to determine the genotype of the individual. The ability of the methods to interrogate particular sites also find value for identification purposes, including for example, forensic and paternity cases. The methods also have utility in detecting the presence of nucleic acids from particular pathogens (e.g., certain viruses, bacteria or fungi).

### III. Determination of Nucleotide at Variant Site

### A. General Approach

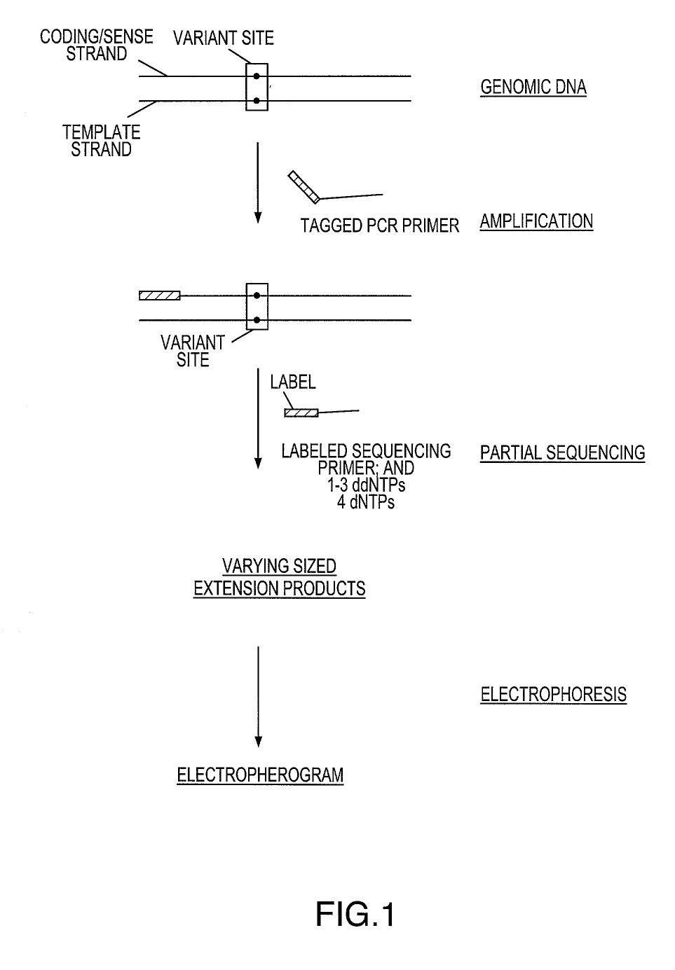

FIG. 1 illustrates the major steps in certain methods of the invention that can be utilized to determine the identity of a nucleotide at a variant site in a target nucleic acid of interest. In some methods, the analysis of which nucleotide is present at a variant site begins with the amplification of the target nucleic acid of interest. The term target nucleic acid refers to single- or double-stranded nucleic acids that include the variant site being interrogated. If double-stranded, the target nucleic acid includes a strand that serves as the template during sequencing reactions (the template strand) and a complementary strand, also referred to as the coding strand, sense strand or replicated strand. As noted above, for double-stranded DNA, the variant site includes the base at the site being interrogated and the complementary nucleotide in the complementary strand. Polymorphisms generally are identified by the nucleotide at the variant site of the coding strand rather than the template strand. Thus, an A/G polymorphism means that in one allelic form an A occupies the variant site of the coding strand; in the other allelic form, G occupies the variant site of the coding strand. Amplification is a useful preliminary step if the nucleic acid is present at low levels, as amplification can increase signal-to-noise ratios. Amplification is also a useful way to introduce a specific primer sequence into the amplicon that is later sequenced.

Amplification is performed using upstream and downstream primers that flank the variant site and can be performed using a variety of known methods (see infra). The primers can be selected to generate amplification products of a variety of different sizes. However, typically the amplification primers are selected to generate amplicons that are 100 to 500 bases long, and in other methods typically 100 to 200 bases long. As shown in FIG. 1, amplification can be conducted with a tagged primer. The tag or nucleic acid segment attached to the primer is not complementary to the nucleic acid being amplified. As the nucleic acid is amplified, however, the tag becomes incorporated into the amplified target nucleic acid. In certain methods, the tag or segment is selected based on its ability to function as a good binding site for the sequencing primers. By introducing the same tag or primer binding site into all the target nucleic acids to be analyzed, the partial sequencing reactions can be conducted using the same sequencing primers, thereby minimizing the number of alterations between analyses and simplifying the overall analysis. For example, it is not necessary to prepare different primers specific for each of the different target nucleic acids to be investigated. Instead, the same sequencing primers can be utilized to sequence multiple different target nucleic acids. Extension products from the different target nucleic acids can be distinguished, for example, by using different labels on the primers used to sequence the target nucleic acids. This can significantly increase sample throughput and reduce the complexity of each analysis.

A variety of different tags can serve as sequencing primer binding sites. Suitable sequences include the M13 universal primer sequence (5′TGTAAAACGACGGCCAGT-3′), the T7 universal primer sequence (5′TAATACGACTCACTATAGGG-3′), the T3 universal primer sequence (5′ATTAACCCTCACTAAAGGGA-3′), the SP6 universal primer sequence (5′ATTTAGGTGACACTATAG-3′) and custom designed primers. A variety of other custom tags or sequences can be used. Such sequences are designed to minimize hybridization of the primer at an unintended sequence.

The target nucleic acid (or amplified target nucleic acid) is sequenced by a chain termination method in which only one to three non-extendible nucleotides are utilized in the sequencing reactions. The sequencing reactions are, however, conducted in the presence of all the deoxynucleotide triphosphates, namely DATP, dTTP, dCTP and dGTP, as well as the appropriate primers and a polymerase. The particular non-extendible nucleotide(s) utilized in the sequencing reactions is (are) selected from those that are complementary to the bases that potentially occupy the variant site of the nucleic acid that serves as the template during the sequencing reaction. For example, if the nucleotide at the variant site of the template strand of the target nucleic acid is either T or G, then the sequencing reaction(s) is/are performed with non-extendible analogs of A and/or C.

A “non-extendible nucleotide” refers to a nucleotide analog that once incorporated into the primer cannot be extended further by a polymerase, i.e., the polymerase is unable to catalyze the attachment of another nucleotide to the 3′ hydroxyl group of the non-extendible nucleotide. Thus, suitable non-extendible nucleotides include nucleotides in which the 3′ hydroxyl group is substituted with a different moiety such that another nucleotide cannot be joined to the non-extendible nucleotide once it is incorporated into a primer. Such moieties include, but are not limited to, —H, —SH and other substituent groups. Specific examples of non-extendible nucleotides include dideoxynucleotides and arabinoside triphosphates.

The partial sequencing reactions are template-dependent extension reactions in which a primer that hybridizes to a target nucleic acid (sometimes to the primer binding site introduced during amplification) is extended to varying degrees depending upon when a non-extendible nucleotide is incorporated into the growing extension product. Consequently, extension products of differing lengths are formed. Unlike certain sequencing reactions, however, the methods of the invention generate only a subset of the standard extension/sequencing products because only a subset of all the non-extendible nucleotides are utilized. If the non-extendible nucleotide supplied is complementary to the nucleotide at the variant site of the target nucleic acid, then one of the extension products formed is a “characteristic extension product” or “characteristic extension fragment”. The characteristic extension product or fragment is the extension product formed when a non-extendible nucleotide is incorporated at the variant site of the extension product (i.e., the site on the extension product that is complementary to the variant site of the nucleic acid serving as template). Consequently, detection of a characteristic extension product serves as an indicator of the nucleotide at the variant site of a target nucleic acid. General methods for conducting sequencing reactions are discussed, for example, by Sanger et al. (see, e.g., Proc. Nat'l Acad. Sci. USA 74:5463-5467 (1977)), which is incorporated herein by reference in its entirety.

The partial sequencing methods of the invention are also distinct from the SBPE approaches sometimes used to analyze nucleic acids. The SBPE reactions generate an extension product of a single size, resulting from incorporation of a single nucleotide to the 3′ end of a primer. As just noted, however, the methods of this invention generate a plurality of different sized fragments. As discussed in additional detail below, these fragments can be used to establish a fingerprint region to rapidly identify which signal in a trace resulting from size-separated extension products (e.g., an electropherogram) corresponds to the characteristic extension product. The relative magnitude of the characteristic extension product and neighboring signals in the trace can be used to determine the identity of the base occupying the variant site and to determine the genotype of a diploid organism for that particular site.

The fact that the methods of the invention involve partial sequencing reactions rather than simply a single base pair extension can also be useful when variant sites are spaced relatively close together [i.e., within the length of the region that can be sequenced during a single sequencing reaction (e.g., within 500 bases of one another)] and at least one of the possible nucleotides at each site is the same. Because the methods of the invention generate characteristic extension products for each variant site, multiple variant sites can be interrogated with a single reaction. Such analyses can be conducted using partial sequencing reactions conducted with only a single nucleotide (although partial sequencing reactions performed with two or three non-extendible nucleotides can also be utilized).

It should also be appreciated that for double-stranded nucleic acids either strand of the nucleic acid can be sequenced. Since the nucleotides at the variant site are complementary, the identity of the nucleotide occupying the variant site of one strand can be used to readily establish the identity of the nucleotide at the variant site of the other strand.

As described more fully below, the sequencing or extension reactions are typically performed using labeled sequencing primers to permit facile detection of the extension products. Nonetheless, in certain other methods the non-extendible nucleotide rather than the sequencing primers is labeled. In some instances, however, labeled nucleotides can interfere with incorporation into the extension products and can make the polymerases more error prone, resulting in the misincorporation of nucleotides into the extension product (see, e.g., Lee, L. G., et al., Nucleic Acids Res. 20:2471-2483 (1992); and Ansorge, W., et al., Methods Mol. Biol. U.S.A. 23:317-356 (1993)). In such instances, it is preferred to utilize labeled sequencing primers.

The sequencing reactions are typically designed so that the 3′ end of the sequencing primer once annealed to the template is at least 10 or 25 nucleotides upstream (i.e., 5′) from the variant site. In other methods, the 3′ end is at least 50 nucleotides from the variant site. If the target nucleic acid is amplified prior to the sequencing reactions, the location of the primer binding site relative to the variant site and the overall length of the target nucleic acid being sequenced can be controlled by the judicious selection of the amplification primers (see supra). In general, the sequencing reactions generate extension products that are 50 to 500 nucleotides in length, and more typically 50 to 200 nucleotides in length.

In certain methods, the sequencing reactions are conducted using thermal cycling methods to increase the amount of extension product formed. These cycling methods involve repeating a cycle including the steps of: annealing a sequencing primer to the template strand, extending the annealed primer, and dissociating extension product by heating the resulting duplex nucleic acid to generate free template strand for another round of annealing, extension and dissociation. Typically, 10 to 40 cycles are performed, more typically 25 to 30 cycles are conducted. A thermocycler is generally utilized to regulate the temperature and thus optimize the annealing and dissociation conditions. For a discussion of thermocycling to conduct primer extension reactions see, for example, Proudfoot, et al., Science 209:1329-1336 (1980) and Sambrook et al., Molecular Cloning: A Laboratory Manual, 2nd edition, Cold Spring Harbor Laboratory Press (1989), both of which are incorporated herein by reference in their entirety.

A number of different polymerases can be used in the sequencing stage of the methods. Suitable polymerases for general sequencing include, for example, TaqFs (PE Applied Biosystems). For thennocycling, suitable polymerases include TaqFs, HotStartTaq DNA polymerase (Qiagen) and OmniBase Sequencing Enzyme (Promega).

Once sequencing extension products have been formed, the products are typically separated. The products can be separated in a variety of ways; however, since the extension reactions generate different sized fragments, size separating the products is a facile way to separate the products. Generally, the products are separated by gel electrophoresis, particularly capillary gel electrophoretic methods using established techniques (see, e.g., Sambrook et al., Molecular Cloning: A Laboratory Manual, 2nd edition, Cold Spring Harbor Laboratory Press (1989)). Other size separation techniques such as high performance liquid chromatography, for example, can also be used. Apparatus for conducting such separations on a microscale (e.g., less than 10 μl) are commercially available and include, for example, the “MegaBace 2000” (Molecular Dynamics, Sunnyvale, Calif.). If extension reactions are conducted in multiple reaction vessels, generally the extension products are pooled prior to separating, so long as products in the different reaction vessels include some distinguishing characteristic (e.g., different labels). Ions in the sample can be precipitated prior to conducting electrophoresis by ethanol precipitation, for example.

The separated extension products are then detected to determine which, if any, characteristic extension products are formed. As noted above, the particular characteristic extension product(s) formed is (are) an indicator of the identity of the nucleotide occupying the variant site of the target nucleic acid. The method of detection depends upon the nature of the label used. For example, if the label is a fluorophore, the detection method uses a detector capable of detecting fluorescence; if the label is a radiolabel, then a device or means for detecting radioactivity is utilized (e.g., photographic films). Often a trace of signals from the size-separated extension products is obtained (e.g., an HPLC plot or an electropherogram).

As noted above, a trace of the size separated extension products can be used to establish a fingerprint for the region surrounding the characteristic extension product. This fingerprint can be used to rapidly identify the region at which the characteristic extension product should appear. Typically, the fingerprint region includes 5-20 signals on either side of the location at which the characteristic extension product appears or should appear, although larger or smaller regions (e.g., about 10 signals on either side of the characteristic extension product) can be used. If the size chosen does not yield a distinctive fingerprint, then a wider region can be examined.

As described in greater detail below, although there can be some variation in peak height along a sequencing trace, the relative height of a given peak (e.g., the peak for the characteristic extension product) compared to its neighbors is relatively constant from sample to sample and tends to be determined by local sequence context. The size of the peak for the characteristic extension product depends upon whether the sample is from a heterozygote or a homozygote. In general, signal magnitude for a heterozyogote is only about half that for adjacent signals in the fingerprint region (the other signals tend to correspond to sites at which the identity of the nucleotide at that site on both copies of the target nucleic acid are the same). Only a half height signal is obtained, because for a heterozygote only half of the copies of the target nucleic acid obtained from the diploid organism generate characteristic extension product. Thus, by comparing the relative signal magnitude for the characteristic extension product with neighboring signals in a trace, genotype and the identity of the nucleotide at the variant site can be rapidly determined. This can be done using one, two or three nucleotides in the partial sequencing reactions (see Examples infra).

### B. Analysis Using a Single Non-Extendible Nucleotide

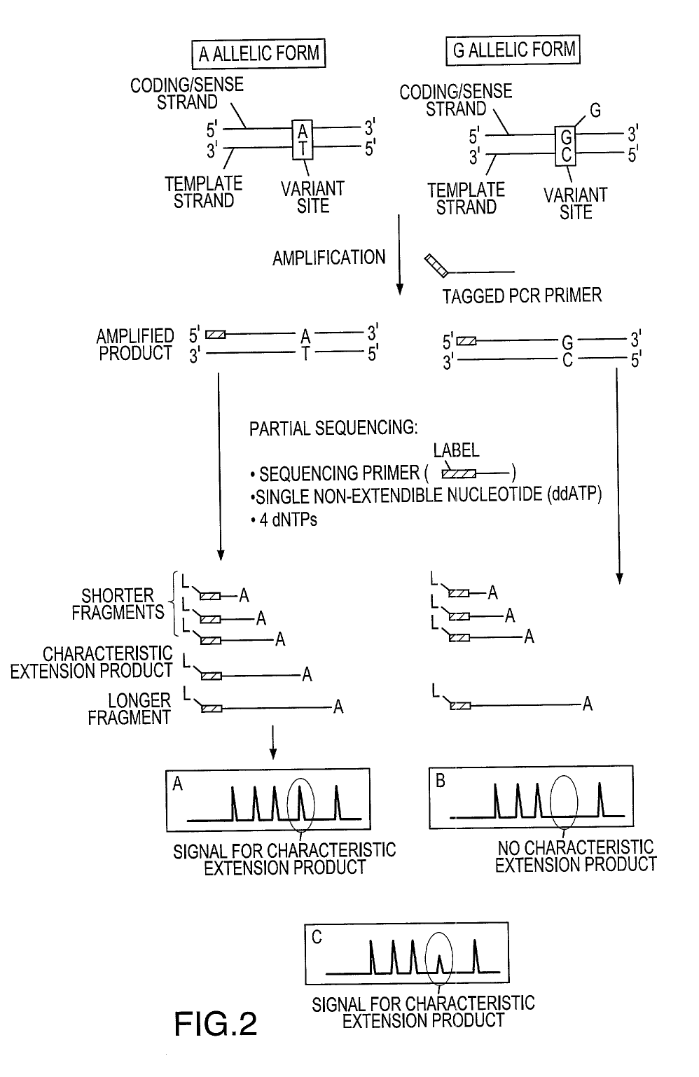

An example of a method for conducting partial sequencing reactions to determine the identity of a base occupying the variant site of a target nucleic acid using a single non-extendible nucleotide is illustrated in FIG. 2. This example illustrates certain steps of the method for a variant site in which the strand to be replicated (i.e., the coding stand) includes either A or G (e.g., an A/G polymorphism). The variant site on the template strand includes the complementary bases T or C, respectively. These particular bases are only exemplary; the variant site can include any other combination of bases as well.

As described above, the method begins with an optional amplification of the target nucleic acid. In the particular example shown in FIG. 2, the upstream amplification primer includes a tag or segment that is not complementary to the target nucleic acid (the tag is represented by the dashed segment). During the amplification reaction (e.g., polymerase chain reaction (PCR)), the tag segment becomes incorporated into the amplicon or amplification product. A useful tag is the M13 universal primer, but as described supra, the tag can include any of a number of other sequences that are non-complementary to the target nucleic acid.

In the partial sequencing step, the sequencing or replication reaction mixtures include the amplicon, a labeled sequencing primer (e.g., fluorescently labeled) complementary to a 3′ segment of the template strand (typically the universal primer sequence or a segment thereof), the four standard dNTPs (DATP, dTTP, dCTP, dGTP) and a single non-extendible nucleotide that is complementary to one of the bases potentially occupying the variant site of the template strand. Polymerase is injected into the reaction mixture to initiate the primer extension reaction. Since the nucleotide at the variant site of the nucleic acid strand serving as template in this example is T or C, then the non-extendible nucleotide is selected to be A or G.

In the example shown in FIG. 2, the particular non-extendible nucleotide chosen is A. For the target nucleic acid including the complementary base T at the variant site of the template strand (or A in the coding stand), the sequencing reactions generate a series of extension products of varying sizes that all include the non-extendible nucleotide A at the 3′ end. One of the extension products is the characteristic fragment corresponding to an extension product in which the non-extendible nucleotide A is incorporated at the variant site of the extension product. Once separated by size (e.g., by capillary gel electrophoresis), the fluorescently labeled extension products can be detected and used to generate an electropherogram in which a series of peaks corresponding to labeled extension product, including the characteristic extension product, are depicted. In this example, detection of a peak for the characteristic extension product means that the variant site includes A in the coding strand or extension product and T in the template strand.

With continued reference to FIG. 2, if the variant site of the target nucleic acid includes a non-complementary base (i.e., any base other than T; the base C in this particular example), then the same series or ladder of extension products is formed as for the other allelic form, with one exception. The exception being that a characteristic extension fragment corresponding to incorporation of A at the variant site of the extension product is not generated since the variant site of the target nucleic acid in this instance includes C rather than T in the template strand. Thus, a peak corresponding to the characteristic extension product does not appear in the electropherogram. All the other peaks, however, are the same as for the target nucleic acid in which the complementary base T is at the variant site of the nucleic acid functioning as template.

This example illustrates the capability of certain methods of the invention to determine which of two allelic forms is present in a target nucleic acid by conducting partial sequencing reactions with a single non-extendible nucleotide selected to be one of the possible nucleotides known to occur at the variant site of the target nucleic acid of interest. If the non-extendible nucleotide used to conduct the sequencing reactions is complementary to the nucleotide at the variant site of the template strand, then a positive signal is detected in the electropherogram. Conversely, if the non-extendible nucleotide utilized to conduct the sequencing reactions is not complementary to the base that occupies the variant site of the template strand, then substantially no signal is detected. The phrase “substantially no signal” meaning that there is no appreciable signal above the background noise level.

Thus, for nucleic acids in which the potential identities of the base at the variant site are known, the absence of signal is essentially a positive result with the approach just described. Since the sequence around the variant site is known, one can verify that all the other expected peaks in the electropherogram are present. So long as they are present, then the absence of a peak at the location at which a signal for the characteristic extension product should appear is strong evidence that the variant site of the nucleic acid functioning as the template lacks a base that is complementary to the non-extendible nucleotide used to conduct the sequencing reaction.

### C. Analysis Utilizing Two Non-Extendible Nucleotides

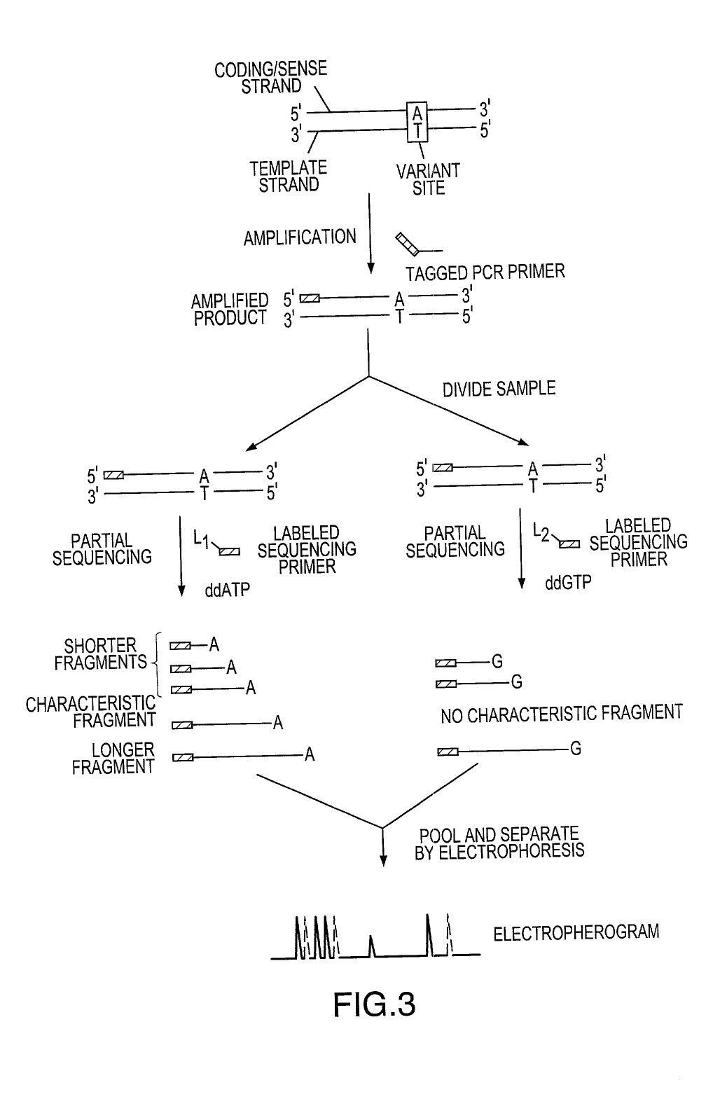

An example of a method utilizing two sequencing reactions with two non-extendible nucleotides is depicted in FIG. 3. The target nucleic acid is the same as the target nucleic acid in the example illustrated in FIG. 2. Although in this particular example, the method is described only for the A allelic form. The results obtained when both allelic forms are present is discussed infra in the section on genotyping. The target nucleic acid can optionally be amplified in these methods, too. The same amplification considerations described in relation to FIG. 1 apply to this example as well.

Certain aspects of the sequencing reaction are the same as described for the sequencing methods utilizing a single non-extendible nucleotide. For example, the nucleotides added to the sequencing reactions are selected to be complementary to the bases known to potentially occupy the variant site of the target nucleic acid. In the particular example depicted in FIG. 3, since the potential bases that occupy the variant site of the template strand are T and C (A and G in the coding strand), the two nucleotides chosen to conduct the sequencing reactions are A and G.

Unlike the methods utilizing a single non-extendible nucleotide, however, the target nucleic acid (or amplified target nucleic acid) is split between two reaction vessels. Into each of the two reaction vessels is added a labeled primer, the four dNTPs (i.e., dATP, dTTP, dGTP and dCTP), and one of the two non-extendible nucleotides selected to conduct the sequencing reaction (in this example, A or G). The components in the two reaction vessels differ in that different labeled primers are added to the two reaction vessels and the non-extendible nucleotide in one reaction vessel is A, whereas the non-extendible nucleotide in the second reaction vessel is G. Sequencing reactions are initiated with the injection of polymerase. The primers in the two reaction vessels generally bear different labels (represented by the symbol L1 and L2) so that the extension products formed in each of the reaction vessels can be readily distinguished.

As shown in FIG. 3, a series of extension products is formed in each reaction vessel. The extension products differ because in one reaction vessel, the various extension products are formed by termination of the extension reaction upon incorporation of the non-extendible nucleotide A. In the second reaction vessel, in contrast, the extension products are formed by termination of the extension reaction upon incorporation of the non-extendible nucleotide G. The extension products also differ in that in the first reaction vessel a characteristic extension product is generated during the sequence reaction. Characteristic extension product is not formed in reaction vessel two because the non-extendible nucleotide is not complementary to the base occupying the variant site of the nucleic acid that serves as template during the sequencing reaction.

The extension products formed via the sequencing reactions are typically pooled and separated by size. The pooled fragments can be separated in a single lane of an electrophoretic gel or within a single capillary. The reaction products formed in the two reaction vessels can be distinguished by the different labels attached to the different primers utilized in the separate reactions. Hence, the various fragments can be detected by detecting signal from the first label borne by extension products generated in the first reaction vessel and detecting signal from the second label borne by extension products generated in the second reaction vessel. In FIG. 3, this is indicated by showing peaks correlated with extension product formed in reaction vessel 1 with solid lines, whereas signals for extension product generated in reaction vessel two are represented with dashed lines.

If the extension products are not pooled and run separately on a gel, it is not necessary to use different labeled primers in the two reaction vessels. In this approach, the different reaction products are discriminated by running the reaction products in different gels or gel lanes. This approach is somewhat more cumbersome and can introduce variability into the results because of variations between electrophoretic runs. The approach described above is typically used because it allows for a quicker and more consistent analysis. In the approach described above, if all the extension products are fluorescently labeled, for example, they can all be detected in a single gel lane simply by changing the wavelength being monitored.

This example illustrates how partial sequencing reactions conducted with just two nucleotides can clearly identify which of two nucleotides is present at the variant site of a target nucleic acid. As with the analyses conducted with a single non-extendible nucleotide, even the absence of a peak can provide a clear indication of the identity of the base occupying the variant site, because the adjacent peaks serve as positive controls. As described further below in the section on genotyping, the method can yield positive signals for diploid organisms that have both allelic forms (i.e., that are heterozygotes). The single non-extendible nucleotide methods, in contrast, generate a positive signal for only one allelic form (see FIG. 2 and accompanying discussion). As just noted, however, even the absence of a signal with the methods of the invention is strong evidence for the absence of an allelic form because the other signals function as controls.

### D. Analyses Conducted with Three Non-Extendible Nucleotides

Certain methods of the invention involve conducting sequencing reactions with three non-extendible nucleotides. The methods closely parallel those described above, especially the methods utilizing two non-extendible nucleotides. The method is most typically used to analyze target nucleic acids wherein the variant site potentially includes three different bases, i.e., nucleic acids that are triallelic, or nucleic acids that include four allelic forms.

The target nucleic acid can be amplified if necessary to increase the amount of target nucleic acid or as a way to incorporate desired binding sites for sequencing primers into the target nucleic acid (see supra). The sequencing reactions are generally performed by splitting the target nucleic acid into three separate reaction vessels. All of the reaction vessels include all four dNTPs and an aliquot of sample containing target nucleic acid. Each reaction vessel receives a primer bearing a different label so that the different reaction products can be distinguished, as well as a different non-extendible nucleotide. The non-extendible nucleotides utilized are selected to be complementary to the bases that potentially occupy the variant site. Extension reactions are initiated with the injection of polymerase.

During the extension reactions, multiple extension products are formed in each reaction vessel. Characteristic extension product is formed in a reaction vessel if the non-extendible nucleotide is complementary to a base occupying the variant site of the target nucleic acid.

Extension products are typically pooled and then separated by size, usually by gel electrophoresis. As with the two nucleotide methods, all the extension products can be separated on a single lane of a gel or within a single capillary. The different extension products generated in each reaction vessel, including the different characteristic extension fragments, are distinguished by the different labels borne by the extension products from the different reaction vessels. The characteristic fragment(s) generated is an indicator of the nucleotides present at the variant site of the target nucleic acid.

### E. Analyses Using Labeled Non-extendible Nucleotides

In the foregoing methods, reactions are conducted using labeled primers. An alternative is to conduct the foregoing methods using labeled non-extendible nucleotides rather than labeled primers. If labeled nucleotides are used instead of labeled primers in the two and three nucleotide methods, it is not necessary to conduct separate sequencing reactions in separate reaction vessels, so long as different labels are used to label the different non-extendible nucleotides. In the two and three nucleotide sequencing methods described above, separate reactions are necessary in order to track which non-extendible nucleotide is incorporated into the extension product. In these methods, the particular non-extendible nucleotide incorporated into extension product is encoded by using different labeled primers for each sequencing reaction conducted with a different non-extendible nucleotide. If differentially labeled nucleotides are utilized, however, the different non-extendible nucleotides are encoded by the particular label borne by the label. Thus, the identity of the base incorporated at the variant site of the extension product (i.e., the nucleotide at the 3′ end of the characteristic extension product) can be directly determined on the basis of the label attached to the non-extendible nucleotide(s) incorporated at the variant site of the extension product.

In some instances, however, labeled nucleotides can interfere with incorporation of the labeled nucleotide into the growing extension product. Such interference can make the polymerases more error prone and result in misincorporation of nucleotides. If problems of this type are encountered or expected to be a concern, the use of labeled primers such as described above is preferred.

### IV. Genotyping

A diploid organism contains two copies of each gene. A complete genotyping analysis involves the determination of whether a diploid organism contains two copies of the wild type allele (a wild-type homozygote), one copy each of the wild type and mutant allele (i.e., a heterozygote), or contains two copies of the mutant allele (i.e., a mutant homozygote). A sample from a diploid organism can include all the allelic forms of a target nucleic acid. Most typically, single nucleotide polymorphisms (SNPs) consist of two allelic forms, i.e., the variant site includes one of two different nucleotides. The most common allelic forms being A/C, A/G, A/T, C/G, C/T and G/T. As noted above, the different allelic forms refer to the base at the variant site of the coding strand rather than the strand that serves as a template during the sequencing reaction.

The ability to determine whether an organism is homozygous for a particular allele or heterozygous is a useful capability because individuals that are homozygous for a mutant allele associated with a disease are at greater risk than individuals that are heterozygous or homozygous for the wild-type allele. Furthermore, individuals that are homozygous mutants for an allele associated with a particular disease sometimes suffer the symptoms of the disease to a greater extent than heterozygotes.

All of the various partial sequencing methods described supra can be utilized to conduct genotyping investigations to determine not only which base is present at a particular target nucleic acid, but also whether a sample contains other allelic forms of the same target nucleic acid. As described above and illustrated in FIGS. 2-4, the ability to discriminate between allelic forms is a consequence of a feature of the methods wherein if the base occupying the variant site of the target nucleic acid is complementary to the non-extendible nucleotide present in the sequencing reaction then a characteristic extension product is formed. If, however, the base occupying the variant site is not complementary to the variant site, then no characteristic extension product is formed.

Hence, in the case of a sample from a diploid organism homozygous for a particular allele in which both copies of the target nucleic acid include a base at the variant site that is complementary to a non-extendible nucleotide used in the sequencing reaction, both copies produce a characteristic extension product. This extension product generates a signal having magnitude (e.g., peak amplitude or peak area) X. In the opposite situation, in which the sample is obtained from a homozygous organism in which the base occupying the variant site of both copies of the target nucleic acid is not complementary to a non-extendible nucleotide used in the sequencing reaction, then no characteristic extension product is generated from either copy of the target nucleic acid. Consequently, substantially no signal is produced. For a heterozygous organism, however, one copy of the target nucleic acid includes a base that is complementary to a non-extendible nucleotide used in the sequencing reaction, and the other copy of the target nucleic acid lacks such a base. Hence, the signal associated with the characteristic extension product in the heterozygote is approximately X/2. This relationship means that the genotype of an organism having a known sequence except at the variant site can be determined in a single partial sequencing reaction conducted with a single non-extendible nucleotide by virtue of signal size. In certain methods, the relative magnitudes of signals for characteristic extension product and signals for other extension products adjacent to the characteristic extension product in an electropherogram are compared. This general capability is best illustrated with a specific example, such as the example set forth in FIG. 2 wherein the genotype of an organism is determined by conducting partial sequence reactions with a single nucleotide.

In FIG. 2, the two allelic forms of the target nucleic acid are shown. In the A allelic form, the variant site of the coding strand includes A (the variant site of the template strand is occupied by the base T). The other allelic form, the G allelic form in this example, includes G at the variant site of the coding strand (the base C occupies the variant site of the template strand). This is the situation in an A/G polymorphism, for example. As illustrated in FIG. 2, when the sequencing reaction is performed with a non-extendible analog of the nucleotide A, the A allelic form which includes the complementary nucleotide T at the variant site of the template strand, generates characteristic extension product. The signal from the characteristic extension product can be detected in an electropherogram, for example (electropherogram A in FIG. 2). However, the same sequencing reaction conducted with the G allelic form of the target nucleic acid fails to generate characteristic extension product because the nucleotide at the variant site of the template strand (C in this example) is not complementary to the non-extendible nucleotide. Consequently, no peak (at least no peak above background) appears in the electropherogram (electropherogram B in FIG. 2).

Thus, an A/A homozygote in which the nucleotide at the variant site of both copies of the template are complementary to the non-extendible nucleotide gives a spectrum as shown in box A of FIG. 2. This spectrum includes the signal for the characteristic extension product (circled). An G/G homozygote in which the nucleotide at the variant site of both copies of the nucleic acid serving as template is not complementary to the non-extendible nucleotide, in contrast, gives a spectrum such as that shown in box B of FIG. 2. This spectrum lacks a signal for the characteristic extension product. In view of these results with homozygotes, a heterozygote (A/G) yields a spectrum in which the signal for the characteristic extension product is approximately half that of the signals for other extension products (electropherogram C of FIG. 2). A specific example of the single nucleotide approach is described in Example 3 below.

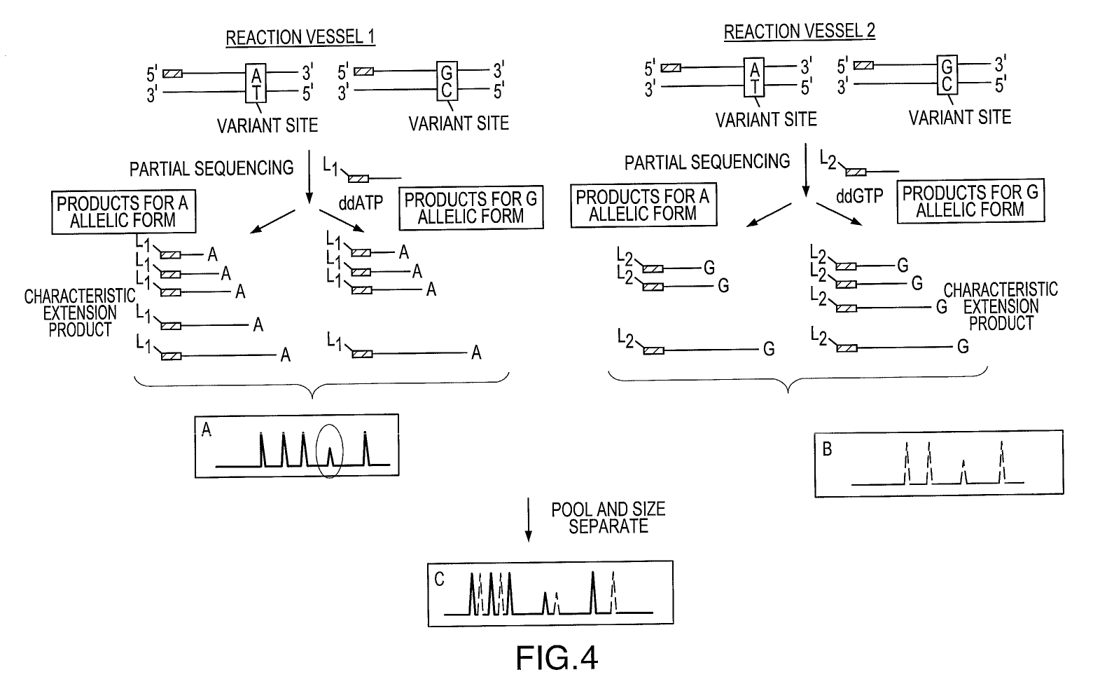

Similar results are obtained when partial sequencing reactions are conducted with two non-extendible nucleotides. This is illustrated in FIG. 4 for an analysis conducted with target nucleic acid obtained from a heterozygote. Hence, when the sample is split and placed into the two reaction vessels, both reaction vessels contain a copy of both allelic forms of the target nucleic acid. The sequencing reactions in the first reaction vessel are conducted in the presence of a non-extendible analog of A (e.g., ddATP). In this reaction vessel, extension products are produced from both allelic forms. However, as shown in FIG. 4, only the A allelic form generates characteristic extension product. If an electropherogram were obtained just for products generated in the first reaction vessel, the result would look like electropherogram A in FIG. 4. The opposite result obtains for partial sequencing reactions in the second reaction vessel that are conducted in the presence of a non-extendible G analog (e.g., ddGTP). In these reactions, the G allelic form generates characteristic extension product, whereas the A allelic form does not. Thus, an electropherogram for products obtained in just this reaction vessel would look like electropherogram B of FIG. 4.

The net result when all the extension products are pooled and size-separated is shown in electropherogram C of FIG. 4. The fluorescent signals from extension products formed in the first reaction vessel are detected at one wavelength (signals indicated with solid lines), and the fluorescent signals for extension products formed in the second reaction vessel are detected at a second wavelength (signals indicated with dashed lines). As expected for a heterozygote, each signal for the characteristic extension product formed in the two reaction vessels is approximately half the magnitude of the signal for other extension products. Collectively, however, the magnitude of the two fractional signals is substantially equivalent to the magnitude for other extension products.

If instead of obtaining the sample from a heterozygote the sample was obtained from an A/A homozygote, the final electropherogram would look like electropherogram C, except that a full height solid signal would be obtained for the peak corresponding to the characteristic extension product. Similarly, if the sample was from a G/G homozygote, the final electropherogram would look like electropherogram C of FIG. 4, but a fall height dashed signal would be obtained. A specific example of a genotyping investigation conducted using the two nucleotide method is described in Example 2.

Hence, as illustrated by these examples, and more specifically Examples 2 and 3 below, the magnitude of the signal for the characteristic extension products can be used to determine whether a sample is obtained from a heterozygote or a homozygote and, if a homozygote, which type of homozygote (i.e., a wild type or mutant homozygote). Typically, the magnitude of the signal for a characteristic extension product is compared to the magnitude of the signal for another extension product formed during the same sequencing reaction as the characteristic extension product. The magnitude of signals for other extension products is generally representative of a signal associated with a homozygote. This is so since the signals for extension products adjacent the signal for a characteristic product in an electropherogram are typically signals for sites that are not polymorphic sites [typically polymorphic sites occur only about once every 1000 bases (Kruglyak, L., Nature Genet., Vol. 17, 21-24(1997); and Collins, F. S., et al., Science, Vol. 278, 1580-1581(1997)].

The reference signal to which the signal for the characteristic extension product is compared can be essentially any signal. The reference signal, however, should be calibrated relative to the magnitude of a signal for extension product generated for either a homozygous or heterozygous condition or some combination thereof. Nonetheless, the most straight-forward approach is simply to compare the signal for the characteristic extension product with the signal for another extension product that appears close to the signal for the characteristic extension product in the electropherogram. This approach has the virtue of simplicity since the signal for the other extension product is detected at essentially the same time as the signal for the characteristic extension product. This approach also minimizes variability in analysis, as the signal for the other extension product is generated under similar conditions and typically is for the same type of label as borne by the characteristic extension product. To further minimize variability, in some methods an average of the magnitude of several signals for extension products is obtained and the signal for the characteristic extension product compared with the average value.

If the genotyping analysis involves comparing the magnitude of the signal for the characteristic extension product (characteristic signal) with the magnitude of the signal for another extension product, one can conclude that the organism from which the sample was obtained is a homozygote if the magnitude of the characteristic signal is substantially equivalent to the signal for another extension product. The particular allelic form is determined by the identity of the non-extendible nucleotide utilized in the extension reaction. If, however, the magnitude of the characteristic signal is approximately half that of the signal for another extension product, then the organism is a heterozygote.

The term “substantially equivalent” means that the difference between the magnitude of the characteristic signal and the magnitude of the signal for the other extension product is less than 30%, in other instances less than 20%, 15%, or 10%, and in still other instances less than 5% or 1%. The percentage difference is calculated by subtracting the magnitude of the signal for the extension product from the magnitude of the characteristic signal and dividing the difference by the magnitude of the signal for the other extension product. The quotient is converted to a percentage and the absolute value obtained. The term “approximately half” or related phrases when used in reference to peak size comparisons means that one peak is between 35% to 65%, more typically 40% to 60% of the peak to which it is being compared. The term “magnitude” when used in reference to signal size can mean simply the signal or peak amplitude, but more typically means the area of a peak (e.g., as recorded).

The comparison process can be computerized. For example, software can be used to compare the magnitude of the characteristic signal and the reference signal (for example, to compute a percentage difference such as described above. The software can also select or provide the appropriate reference signal. Furthermore, the software can be used to calculate an average magnitude for several signals associated with other extension products.

Another feature of the methods illustrated by these examples is that detection of signals for other extension products serves as an internal control. Since the analyses are conducted with nucleic acids for which the sequence is typically known (except for the identity of the nucleotide at the variant site), one knows whether all the expected extension products are in fact formed. With such internal controls, the absence of a signal for a characteristic extension product is strong evidence that the target nucleic acid lacked a base at the variant site that was complementary to the non-extendible nucleotide(s) present in the sequencing reactions rather than simply being a consequence of a problem with the reaction.

### V. Multiplexing

The partial sequencing methods described above can be modified and utilized in multiplexing formats to identify a nucleotide at multiple variant sites in a single reaction. Such formats allow for rapid sequence determinations in many loci and/or individuals simultaneously. Essentially this is achieved by pooling several partial sequencing reactions together. The multiple variant sites can be multiple sites on the same target nucleic acid, such sites being within the same gene or at sites in different genes. Alternatively, the multiple sites can be the same site on target nucleic acids obtained from different individuals, or multiple different sites on target nucleic acids from different individuals.

The multiplexing methods closely parallel the partial sequencing methods described above in which sequencing reactions are performed with one, two or three non-extendible nucleotides. In order to correlate extension products with the target nucleic acid or variant site with which the product is associated, a number of different strategies can be utilized. For example, one strategy is to use different primers for the different variant sites. Typically, the different primers bear different labels to facilitate discrimination between the extension products. An example of this approach is to use different fluorescent labels that fluoresce at different wavelengths.

Another approach is to utilize the same primer to conduct the sequencing reactions for the different variant sites being interrogated, but to segregate the sequencing reactions for the different interrogations into separate reaction vessels. Extension products from the various reaction vessels can be encoded by differentially labeling the primers in the different reaction vessels.

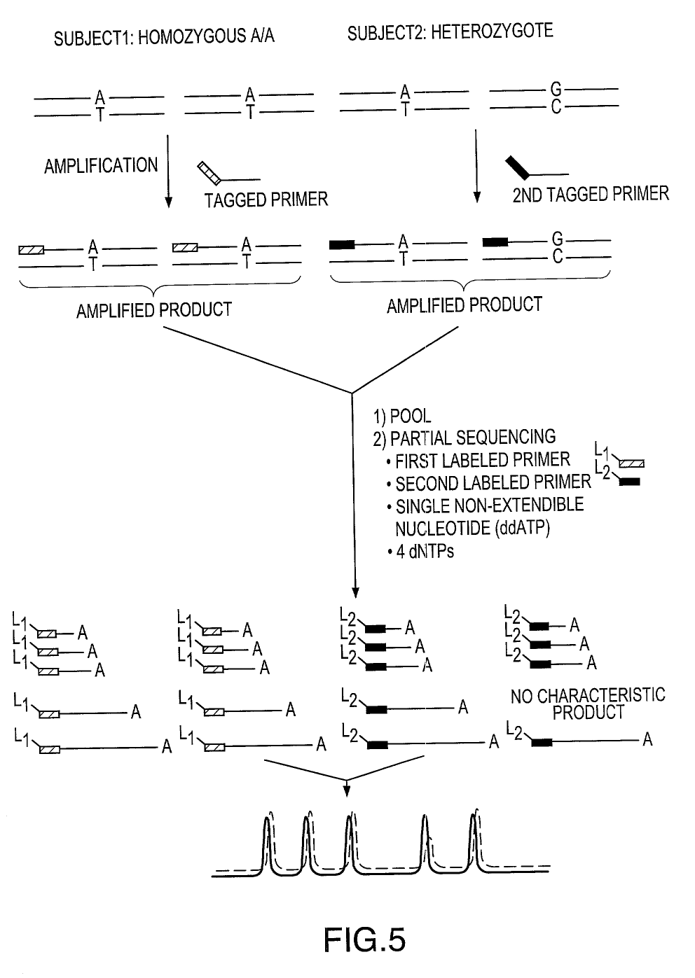

An example of one method for conducting a multiplexing analysis of target nucleic acids from two diploid subjects is shown in FIG. 5. This example utilizes the method wherein partial sequencing reactions are conducted with a single nucleotide. Subject 1 is an A/A homozygote, i.e., both copies of the target nucleic acid have the base A at the variant site of the coding strand. Subject 2 in contrast is a heterozygote. Hence, a sample from this subject contains A and G allelic forms of the target nucleic acid. Initially, the samples from the two subjects are amplified separately. The amplification reactions are conducted using tagged primers, wherein a tag attached to the primer is non-complementary to the target nucleic acid. Different tagged primers are used to amplify the target nucleic acid in the two reactions. The result being that the amplified product from subject 1 includes a segment (e.g., a sequencing primer binding site) that differs in sequence from the segment incorporated into the amplified product from subject 2.

The amplified product is then pooled and partially sequenced using two different sets of primers (L1 and L2 in the example shown in FIG. 5; e.g., different fluorescent labels) in the presence of a single non-extendible nucleotide, in this example ddATP. One primer hybridizes to the segment introduced into the target nucleic acid from the first subject during amplification. Likewise, the second primer hybridizes to the segment introduced during amplification of target nucleic acid from the second subject. The two primers have different labels so that extension product generated from subject 1 can be distinguished from extension product generated from subject 2. Thus, using a combination of primers wherein some selectively hybridize to target nucleic acid from one subject and the other primers to target nucleic acids from another subject and differentially labeling the sets of primers, it is possible to distinguish which subject the extension products correlate with. In the example shown in FIG. 5, extension product from subject 1 bears a first label, while extension product from subject 2 bears label 2.

All the extension products formed are shown in FIG. 5. Since subject 1 is a A/A homozygote and the non-extendible nucleotide is ddATP, characteristic extension product is formed and its intensity is comparable to the intensity of signals for other extension products. In the case of subject 2, a heterozygote, characteristic extension product is formed since there is one copy of the A allelic form. However, the signal magnitude for the characteristic extension product is about half of the signal magnitude of the other extension products. The signals for the extension products shown for subject 2 are shown in dashed lines to indicate that detection is at different wavelength as compared to signals for extension products for subject 1.

Rather than introduce different sequencing primer sites into the target nucleic acid as shown in FIG. 5, the sequencing reactions for subject 1 and subject 2 can be conducted in separate reaction vessels. In this approach, the sequence of the sequencing primers can be the same, so long as the primers have a distinguishing feature (e.g., different labels) that allow extension product in one reaction vessel to be distinguished from extension product generated in the other reaction vessel.

Yet another option is to simply conduct sequencing reactions in different reaction vessels, wherein each reaction vessel contains a different target nucleic acid. Extension products from the different reaction vessels are then sequentially injected, with sufficient time between injections to allow the extension products from different samples to be fully resolved on the capillary column. Thus, multiplexing in this format involves separating extension product by time.

The two base partial sequencing methods can be conducted in an analogous fashion to the various options set forth above.

### VI. Detection

### A. General Options

The sequencing extension products formed, including the characteristic extension products, can be detected in a number of different ways. One general approach is to simply label the extension products after they are formed. Essentially any intercalation dye capable of intercalating into duplex DNA can be used to detect extension product. Specific examples of suitable dyes include, but are not limited to, thiazole orange, ethidium bromide, propidium iodide, chromomycin, acridine orange, Hoechst 33258, Toto-1, Yoyo-1, DAPI (4′,6-diamidino-2-phenylindole hydrochloride), SyberGreen and Pico Green (the latter two dyes being available from Molecular Probes, Inc. of Eugene, Oreg.).

In order to gain more flexibility and to avoid having to label products late in the analysis, more typically labeled primers are utilized to generate labeled extension products. Alternatively, in certain methods, the sequencing reactions are conducted with labeled non-extendible nucleotides. These labeled nucleotides can produce labeled extension product also.

### B. Labels

1. Types

The primer or non-extendible nucleotide includes a label that is either directly or indirectly detectable. The label can be any compound or molecule that can be detected and that does not significantly interfere with the extension reaction (e.g., interfering sufficiently such that an undetectable amount of extension product is formed and/or causing elevated rates of misincorporation such that an accurate determination of the identity of the nucleotide at the variant site is not possible). Suitable labels include, but are not limited to, fluorophores, chromophores, molecules that emit chemiluminescence, magnetic particles, radioisotopes, mass markers, electron dense particles, enzymes, cofactors, substrates for enzymes and ligands having specific binding partners (e.g., avid/biotin).

Certain methods utilize fluorescent molecules as the labels, as a number of commercial instruments have been developed for the detection of fluorescently labeled nucleic acids. A variety of fluorescent molecules can be used as labels including, for example, fluorescein and fluorescein derivatives, rhodamine and rhodamine derivatives, naphythylamine and naphthylamine derivatives, cyanine and cyanine derivatives, benzamidizoles, ethidiums, propidiums, anthracyclines, mithramycins, acridines, actinomycins, merocyanines, coumarins, pyrenes, chrysenes, stilbenes, anthracenes, naphthalenes, salicyclic acids, benz-2-oxa-1-diazoles (also called benzofurazans), fluorescamines and the Bodipy series of dyes (Molecular Probes, Inc.).

In some instances, primers include an energy transfer dye pair consisting of a donor and acceptor dye sufficiently close to one another so that the donor, once excited, can transfer energy to the acceptor dye. These dyes are capable of increasing emission intensities. One group of donor and acceptor dyes includes the xanthene dyes, such as fluorescein dyes, and rhodamine dyes. A variety of derivatives of these dyes are commercially available. Often functional groups are introduced into the phenyl group of these dyes to serve as a linkage site to an oligonucleotide. Another general group of dyes includes the naphthylamines which have an amino group in the alpha or beta position. Dyes of this general type include 1-dimethylaminonaphthyl-5-sulfonate, 1-anilino-8-naphthalende sulfonate and 2-p-touidinyl-6-naphthalene sulfonate. Other dyes include 3-phenyl-7-isocyanatocoumarin, acridines, such as 9-isothiocyanatoacridine and acridine orange, pyrenes, bensoxadiazoles and stilbenes. Additional dyes include 3-(ε-carboxypentyl)-3′-ethyl-5,5′-dimethyloxa-carbocyanine (CYA), 6-carboxy fluorescein (FAM), 5&6-carboxyrhodamine-110 (R110), 6-carboxyrhodamine-6G (R6G), N′,N′,N′,N′-tetramethyl-6-carboxyrhodamine (TAMRA), 6-carboxy-X-rhodamine (ROX), 2′, 4′, 5′, 7′, -tetrachloro-4 -7 -dichlorofluorescein (TET) and 2′, 7′-dimethoxy -4′, 5′-6 carboxyrhodamine (JOE), ALEXA, Cy3 (C3 thiacarbocyanine) and Cy5 (C5 oxadicarbocyanine).

Further guidance regarding the selection of donor and acceptor pairs that can effectively be used with the methods of the present invention include: Fluorescence Spectroscopy (Pesce et al., Eds.) Marcel Dekker, New York, (1971); White et al., Fluorescence Analysis: A Practical Approach, Marcel Dekker, New York, (1970); Berlman, Handbook of Fluorescence Spectra of Aromatic Molecules, 2^{nd} ed., Academic Press, New York, (1971); Griffiths, Colour and Constitution of Organic Molecules, Academic Press, New York, (1976); Indicators (Bishop, Ed.). Pergamon Press, Oxford, 19723; and Haugland, Handbook ofFluorescent Probes and Research Chemicals, Molecular Probes, Eugene (1992).

Another class of dyes that can be utilized are infrared dyes.

2. Attachment of Label to Primer

Attaching a label to a primer can be accomplished in a number of different ways. One general approach involves preparing derivatives of dyes that contain appropriate functional groups for linking the dyes to the sequencing primer. Such methods are described, for example, by Marshall, Histochemical J. 7:299-303 (1975); Mechnen et al. in U.S. Pat. No. 5,188,934; Bergot et al. in PCT publication PCT/US90/05565; Ullman et al. in U.S. Pat. No. 3,996,345 and Khanna et al. in U.S. Pat. No. 4,351,760.

In another approach, a label is linked to a nucleotide in the sequencing primer via a linker. A number of such linkers are commercially available and have varying lengths. Such linkers are useful for obtaining a desired distance between the primer and label to ensure that the label does not interfere with the extension reactions. In general such linkers include a flnctional group (e.g., amino, hydroxyl, sulfhydryl, carboxyl) at each end so that one end can be attached to a nucleotide in the sequencing primer and the other end attached to the label (e.g., fluorescent molecule). Examples of such linkers include “Amino Modifier C3”, “Amino Modifier C6,” “Amino Modified C7” and “Amino Modified C12” that are available from Operon Technologies, Inc. Another suitable linker is the “Uni-Link Amino Modifier” available from Clonetech (Palo Alto, Calif.).

Alternatively, modified nucleotides designed to allow for attachment of a label can be incorporated into the sequencing primer. Examples of such modified nucleotides include, for example, 5′-dimethoxytrityl-5-[N-(trifluoroacetylaminohexyl)-3-acrylimido]-2′-deoxyuridine,3′-[(2-cyanoethyl)-(N,N-diisopropyl)]-phosphoramidite, marketed under the name “Amino-Modifier C6 dT” and the related modified nucleotide “Amino Modifier C2 dT,” both available from Glen Research (Sterling, Va.) and designed to function as modified thymidine nucleotides. These molecules contain a protected primary amine that can serve as the attachment site of a label (e.g., a fluorescent label) following deprotection. Methods for incorporating such modified nucleotides into a primer are described, for example, in U.S. Pat. Nos. 5,654,419; 5,688,648; 5,853,992; and 5,728,528 to Mathies et al. and Glazer et al. Single dye labeled R110-ddNTPs, R6G-ddNTPs, TAMRA-ddNTPs and ROX-ddNTPs can be purchased from DuPont NEN (Boston, Mass.).

### VII. Primers

Primers (e.g., the sequencing primers) can be either naturally occurring nucleic acids or prepared using synthetic methods. If synthesized, the primers can be synthesized either enzymatically in vitro, enzymatically in vivo or non-enzymatically in vitro.

The primers are sufficiently long to specifically bind to the appropriate target nucleic acid and to form a stable hybridization complex under the extension reaction conditions. Typically, the primers are 15 to 30 nucleotides in length; in other instances, the primers are 20 to 24 nucleotides long. The length of the primers can be adjusted to be longer or somewhat shorter depending upon the particular sequence to which they hybridize (e.g., primers with a high G/C content typically can be shorter than those with a low G/C content).

As noted above, the 3′ end of a sequencing primer once annealed to a template typically is at least 10, 20, 30, 40 or 50 nucleotides from the variant site. Typically, the primers are designed to be perfectly complementary over their entire length with the template, although a certain number of mismatches can be tolerated so long as specificity in hybridization is not sacrificed.

If labeled, the label is typically attached to or near the 5′ end, although the label can be attached at more internal locations. The goal is to attach the label at a location so that the label does not significantly interfere with the activity of polymerase and/or cause misincorporation. Methods for labeling primers is described supra in the detection section.

In certain methods, the primer can include one or more moieties that allow for the affinity separation of the extension product or primer from unincorporated reagents and/or the target nucleic acid and/or other nucleic acids in the test sample. For example, the primer can include biotin which permits the affinity separation of the primer or extension product from other reaction components through binding of biotin to streptavidin molecules attached to a solid support. As another example, a support can be attached to a nucleic acid sequence that is complementary to the primer or extension product generated therefrom. Hybridization between the primer and its complementary sequence also allows for affinity separation.

### VIII. Samples

### A. Types of Target Nucleic Acids

The methods of the present invention can be utilized to determine the identity of a nucleotide at a variety of different types of variant sites including, but not limited to, SNPs and mutations such as transitions, transversions, insertions and deletions. The presence or absence of a target nucleic acid in a sample can be detected generally as the presence or absence of a particular nucleotide. Individual nucleotides located at a particular site can also be identified by the methods described herein.

The methods presented are generally applicable to deoxyribonucleic acids, ribonucleic acids, or copolymers thereof. The nucleic acids can be single-stranded or double-stranded. The target nucleic acid can include non-naturally occurring nucleotide analogs including, for example, deoxyinosine or 7-deasa-2-deoxyguanosine. Such analogs destabilize duplex DNA and allow a primer annealing and extension reaction to occur in double-stranded nucleic acids without completely separating the two strands. In some instances, RNA samples are first reversed transcribed to form cDNA before use.

The target nucleic acid can be only a fraction of a larger nucleic acid or can be present initially as a purified and discrete molecule. Additionally, the target nucleic acid can constitute the entire nucleic acid or can be a fraction of a complex mixture of nucleic acids. The target nucleic acid can be synthesized enzymatically in vivo, synthesized enzymatically in vitro, or synthesized non-enzymatically.

### B. Sources

The target nucleic acid can be from any source. The samples that include the target nucleic acids can be natural or synthetic using enzymatic or synthetic organic techniques. Likewise, the sample can be taken from any organism, including but not limited to, plants, microorganisms (e.g., bacteria, fungi and viruses), vertebrates, invertebrates and mammals (e.g., humans, primates, horses, dogs, cows, pigs and sheep).

For assay of genomic DNA, virtually any biological sample (other than pure red blood cells) is suitable. Samples can be obtained from the tissues or fluids of an organism; samples can also be obtained from cell cultures, tissue homogenates or synthesized as described above. For example, samples can be obtained from whole blood, serum, semen, saliva, tears, urine, fecal material, sweat, buccal, skin, spinal fluid and hair. Samples can also be derived from in vitro cell cultures, including the growth medium, recombinant cells and cell components. For assay of cDNA or mRNA reverse transcribed to form cDNA, the tissue sample is obtained from an organ in which the target nucleic acid is expressed. For example, if the target nucleic acid is a cytochrome P450, the liver is a suitable source. Samples for use in prenatal testing can be obtained from amniotic fluid.

The target nucleic acid(s) can also be obtained from non-living sources suspected of containing matter from living organisms. For example, in the instance of samples obtained for forensic analysis, the target nucleic acids can be obtained from samples of clothing, furniture, weapons and other items found at a crime scene.

### C. Sample Preparation

In some instances, the samples contain such a low level of target nucleic acids that it is useful to conduct a pre-amplification reaction to increase the concentration of the target nucleic acids. If samples are to be amplified, amplification is typically conducted using the polymerase chain reaction (PCR) according to known procedures. See generally, PCR Technology: Principles and Applications for DNA Amplification (H. A. Erlich, Ed.) Freeman Press, NY, N.Y. (1992); PCR Protocols: A Guide to Methods and Applications (Innis, et al., Eds.) Academic Press, San Diego, Calif. (1990); Mattila et al., Nucleic Acids Res. 19:4967 (1991); Eckert et al., PCR Methods and Applications 1: 17 (1991); PCR (McPherson et al. Ed.), IRL Press, Oxford); and U.S. Pat. Nos. 4,683,202 and 4,683,195, each of which is incorporated by reference in its entirety. Other suitable amplification methods include: (i) the ligase chain reaction (LCR) [see Wu and Wallace, Genomics 4:560 (1989); and Landegren et al., Science 241:1077 (1988)]; (ii) transcription amplification [Kwoh et al., Proc. Natl. Acad. Sci. USA 86:1173 (1989)]; (iii) self-sustained sequence replication [Guatelli et al., Proc. Nat. Acad. Sci. USA, 87:1874 (1990)]; and (iv) nucleic acid based sequence amplification (NASBA), each of which are incorporated by reference in their entirety.

Further guidance regarding nucleic sample preparation is described in Sambrook, et al., Molecular Cloning: A Laboratory Manual, 2^{nd} Ed., Cold Spring Harbor Laboratory Press, (1989), which is incorporated herein by reference in its entirety.

### IX. Utility

The methods of the invention are generally useful for determining the identity of a nucleotide at a variant site. These methods, however, find use in a variety of more specific applications. One use is the identification and detection of point mutations (e.g., somatic point mutations), specifically those mutations known to be correlated with diseases. For example, the methods described herein are useful for identifying whether a nucleic acid from a particular subject includes a wild-type allele or a mutant allele at a particular SNP site. Furthermore, in a single analysis, the methods can be utilized to establish the genotype of the individual being tested (i.e., distinguish whether the individual is a wild type homozygote, a heterozygote or a mutant homozygote).

The genotyping utility of the present methods makes them useful within the context of medical diagnosis and prognosis. Since many SNPs are associated with various diseases, clinicians can utilize the results of the genotype study to assess the presence of disease, whether an individual is a carrier of disease, the likelihood that an individual will get a particular disease and the efficacy of various treatment alternatives.

The methods also have a variety of non-medical uses. Such utilities include detecting pathogenic microorganisms, paternity testing and forensic analysis. The methods can also be used to identify SNPs in non-humans, including for example plants, bacteria and viruses.

These various uses are described more fully below.

### A. Correlation Studies

Use of the methods of the present invention to acquire diagnostic information involves obtaining a sample from a number of different individuals known to have a common disease and conducting screening tests to determine whether they consistently share a common genotype at one or more SNP sites. The results of such screening can be used to establish correlations between certain genotypes and certain diseases.

In a related fashion, the methods of the invention can be used to develop correlations between certain genotypes and patient prognosis. For example, the genotype of a population of individuals suffering from a common disease can be determined at one or more SNP sites. The health history of the individuals can be monitored with time to establish correlations between certain genotypes and disease outcomes.

The methods of the invention can also be used to formulate optimal treatment protocols for a particular disease. The methods described herein can be used to place individuals into groups that share a common phenotype and genotype. The group can then be subdivided into various groups that each receive various forms of treatment. By monitoring the health status of the different treatment groups over time, the most effective treatment program for a particular genotype can be established.

### B. Use of Current Methods as Screening and Therapeutic Tool

In instances in which a correlation between a particular genotype and disease state have already been established, the methods of the invention can be utilized as a diagnostic tool, a prognostic tool and as a means for assessing the success of various treatment options.

For patients having symptoms of a disease, the methods of the present invention can be used to determine if the patient has a genotype known to be associated with a disease that commonly causes the symptoms the patient exhibits. For example, if the genotyping methods of the invention show that the individual has a genotype associated with a particular disease and further that the genotype is associated with poor recovery (e.g., a mutant homozygote), the physician can counsel the client regarding the likely effectiveness of aggressive treatment options and the option of simply foregoing such treatments, especially if the disease is quite advanced. On the other hand, if the genotype is associated with good recovery, the physician can describe a range of treatment options varying from simply monitoring the disease to see if the condition worsens or more aggressive measures to ensure that the disease is attacked before it gets worse.

The methods of the present invention are also valuable for assessing the actual risk of an individual known to be susceptible to acquiring a disease (e.g., an individual coming from a family that has a history of having the disease). By determining whether the individual is a mutant homozygote for the SNP associated with the disease or a heterozygote, a physician can more accurately assess and counsel the patient regarding the likelihood that the patient will begin suffering from disease, factors involved in triggering the disease and the pros and cons regarding different treatment alternatives.

Similarly, certain methods of the invention can also be used to identify individuals at risk for disease, even though they have no symptoms of disease or no known susceptibilities to disease. An individual in this category would generally have no disease symptoms and have no family history of disease. In such cases, the methods of the present invention can be used as a useful preventive screening tool. Using the methods of the present invention, a number of selected SNP sites known to be associated with certain diseases can be interrogated to identify the genotype of the individual at those sites. If a particular genotype were identified that was known to be associated with a particular disease, then a physician could advise the individual regarding the likelihood that the disease would manifest itself and the range of treatment options available.

### C. Examples of Diseases That Can Be Monitored

A large number of diseases have been shown to be correlated with particular allelic forms of SNPs. An extensive list of such SNPs are presented in WO 93/02216 and by Cooper et al. (Hum. Genet. 85:55-74 (1990)), both of which are incorporated herein by reference in their entirety. Specific examples of diseases associated with SNPs include: sickle cell anemia and β-thalassemias (mutation in β-globin gene; Antonarakis, New Eng. J. Med., 320:153-163 (1989)), cystic fibrosis (mutation in cystic fibrosis transmembrane receptor (CFTR); see Kerem, et al., Science 245:1073-1080 (1989)), hyperlipoproteinemia (mutation in apolipoprotein E gene; see Mahley, Science 240:622-630 (1988)), a wide variety of autoimmune diseases (mutations in human major histocompatibility complex; see Thomson, Ann. Rev. Genet., 22:31-50 (1988); Morel et al., Proc. Nat. Acad. Sci. USA, 85:8111-8115 (1988); and Scharf, et al., Proc. Nat. Acad. Sci. USA, 85:3504-3508 (1988)) and the formation of oncogenes (mutations to the human ras-gene family; see, e.g., Bos et al., Nature, 315:726-730 (1985); Farr et al., Proc. Natl. Acad. Sci. USA, 85:1629-1633 (1988); and Neri, et al., Proc. Natl. Acad. Sci. USA, 85:9268-9272 (1988)). Other genes containing SNPs associated with disease include genes encoding for angiotensinogen, angiotensin converting enzyme, cholesterol ester transfer protein, dopamine receptors, serotonin receptors, and HIV reverse transcriptase (RT).

### D. Other Uses

The methods described herein can also be used to identify point mutations in microorganisms that could potentially cause altered pathogenicity or resistance to certain therapeutics. The methods can also be used to identify cells and strains having a desired genetic constitution for use in various biotechnology applications.

The methods described herein can also detect the presence of somatic mutations that can result in various diseases, including cancer for example.

With knowledge gained from the genotyping capabilities of the methods described herein, clinicians can conduct prenatal testing using cells obtained from a fetus to check for a variety of inheritable diseases, such as those diseases associated with the SNPs listed above. The methods can also be used to identify carriers of mutant alleles. Such information can be of use by a couple prior to conception as they evaluate the risks of having a child with certain birth defects or inheritable diseases.

Methods of the invention may also be utilized in various identification applications, such as in the field of forensic medicine or paternal testing. In the case of forensic analysis, polymorphisms in specific genes can be determined in, for example, blood or semen obtained from a crime scene to indicate whether a particular suspect was involved in the crime. In like manner, polymorphism analysis can be utilized in paternity disputes to aid in determining whether a particular individual is the father of a certain child.

In another application, certain methods of the invention are used in blood typing or tissue classification. Tissue classifications, for example, can be determined by identifying polymorphisms specific for a particular individual.

The following examples are provided to illustrate certain aspects of the invention, and should not be construed to limit the scope of the invention.

### EXAMPLE 1

### Experimental Methods

### I. Amplification

Regions of genomic DNA of interest were typically amplified using polymerase chain reaction (PCR). PCR upstream primer containing an extra forward M13 universal primer sequence at 5′ end (5TGTAAAACGACGGCCAGT—) and a downstream primer were designed to amplify about 100 to 160 nucleotides region for each exon. The mutation position was arranged about 30 to 100 nucleotides away from the 5′ end of the upstream primer. Individual exon was amplified using AmpliTaq Gold System (Perkin Elmer). In most instances, 10 μl of PCR reactions were started with 10 ng of genomic DNA and cycled for 35 to 40 times. Exonuclease I (Exo I; 1 unit) and 0.4 unit of Shrimp Alkaline Phosphatase (SAP) in mixture of 0.5 μl was added to 5 μl of PCR amplicon and incubated at 37° C. for 60 min to remove excess PCR primers and dNTPs. After this cleaning process, the mixture was heated at 80° C. for 15 min to de-activate the enzymes.

### II. One Nucleotide Sequencing Reactions

Sequencing reactions were conducted using modified Sanger dideoxy sequencing chemistry with 5′ dye-labeled primers (see, e.g., Proc. Nat'l Acad. Sci. USA 74:5463-5467 (1977), incorporated by reference in its entirety). For one sequencing fragments reaction, only a single reaction was needed for each bi-allelic SNP. One microliter of 5x sequencing reaction buffer, 1 μl of TaqFs (1 unit), 1 μl of 400 nM 5′ dye-primer 1 with M13 universal sequence (5′ Dye-TGTAAAACGACGGCCAGT), 1 μl of ddXTP/dNTP or ddYTP/dNTP mixture and 1 μl of enzyme-cleaned PCR amplicon was added and mixed. ddXTP and ddYTP refer to dideoxynucleotides selected to be complementary to a base potentially occupying the variant site in the nucleic acid serving as template during the sequencing reactions.

Typically, 25 to 30 cycles were performed to generate sufficient extension product. After the sequencing reactions, a solution containing 15 μl of de-ionized water, 2 μl of NaOAC (3 M at pH 5.4) and 50 μl of 95% ethanol were mixed with the pooled reaction. The mixtures were set at room temperature for 20 minutes and then centrifuged at the maximum speed of a micro-centrifuge for 20 minutes. After centrifugation, the tube was inverted and centrifuged at 1000 rpm for 1 minute to completely remove the supernatant. Six microliters of loading solution was added into the tube and vortexed gently to resuspend the precipitated sequencing fragments.

### III. Two Nucleotide Sequencing Reactions

Sequencing reactions were conducted using modified Sanger dideoxy sequencing chemistry with 5′ dye-labeled primers. Two separate reactions were performed for each SNP site. For tube 1, 1 μl of 5x sequencing reaction buffer, 1 μl of TaqFs (1 unit), 1 μl of 400 nM 5′ dye-primer 1 with M13 universal sequence (5′ Dye1-TGTAAAACGACGGCCAGT), 1 μl of ddXTP/dNTP mixture and 1 μl of enzyme-cleaned PCR amplicon was added and mixed. For tube 2, 1 μl of 5x sequencing reaction buffer, 1 μl of TaqFs (1 unit), 1 μl of 400 nM 5′ dye-primer 2 with M13 universal sequence(5′Dye2-TGTAAAACGACGGCCAGT), 1 μl of ddYTP/dNTP mixture and 1 μl of cleaned PCR amplicon was added and mixed. ddXTP and ddYTP refer to dideoxynucleotides selected to be complementary to a base potentially occupying the variant site in the nucleic acid serving as template during the sequencing reactions.

Typically, 25 to 30 cycles were performed to generate sufficient extension product. After reaction, the solution in these two tubes were pooled together. Ten microliters of de-ionized water, 2 μl of NaOAC (3 M at pH 5.4) and 50 μl of 95% ethanol were mixed with the pooled reaction. The mixtures were set at room temperature for 20 minutes and then centrifuged at the maximum speed of a micro-centrifuges for 20 minutes. After centrifugation, the tube was inverted and centrifuged at 1000 rpm for 1 minute to completely remove the supernatant. Six microliters of loading solution was added into the tube and vortexed gently to resuspend the precipitated sequencing fragments.

### IV. Electrophoretic Separation

Electrophoretic separation was performed on the MegaBace 2000 instrument (Molecular Dynamics, Sunnyvale, Calif.). Samples were injected into capillaries at 3000 Volts for 45 seconds. Extension products were separated by conducting electrophoresis at 9000 volts for about 45 minutes. The fluorescence signals associated with labeled extension product were collected.

### EXAMPLE 2

### Single Nucleotide Polymorphism Analysis Utilizing Two Nucleotide Methods

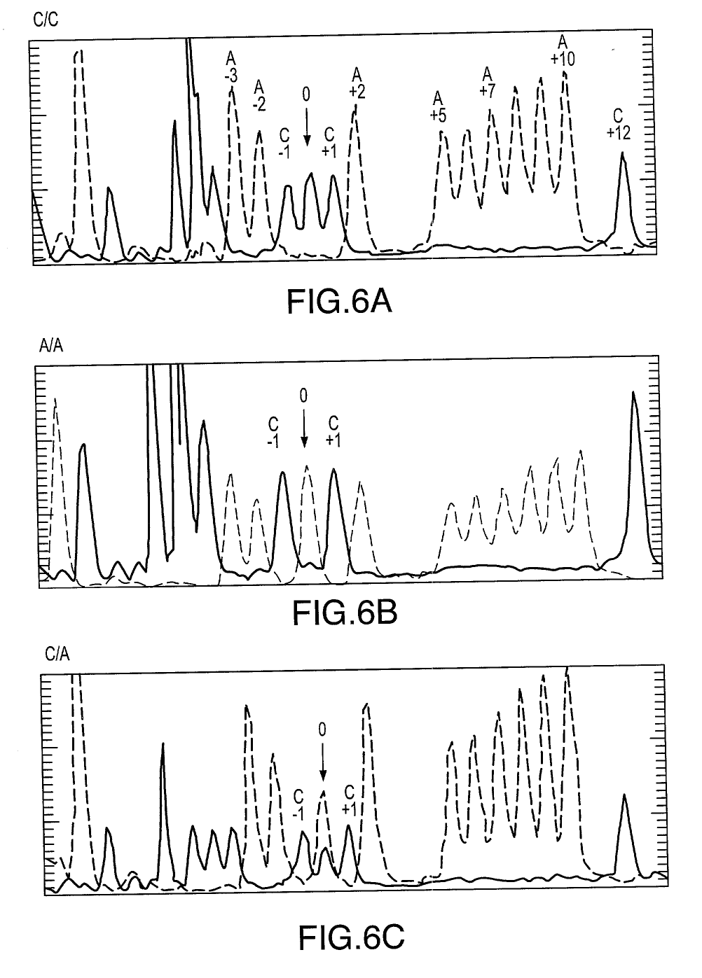

A C/A single nucleotide polymorphism was analyzed using the two nucleotide sequencing reaction approach described in Example 1. The primer utilized in the reaction vessel containing ddCTP was labeled with the fluorescent label FAM (blue emission); whereas, the primer in the sequencing reaction conducted with ddATP was labeled with the fluorescent label JOE (green emission). Electropherograms or traces of labeled extension products as separated by size are shown in FIGS. 6A-C. The dashed lines represent signals originating from extension product into which ddATP was incorporated at the 3′ end (green signal). The solid lines represent signals corresponding to extension product in which ddCTP was incorporated at the 3′ end (blue signal). The bold arrow indicates the signal associated with the characteristic extension product, i.e., extension product generated when the dideoxynucleotide was complementary to the base occupying the variant site of the nucleic acid serving as template for the sequencing reaction.

FIG. 6A is for a sample obtained from a C/C homozygote. As can be seen, the only signal for the characteristic extension product is a blue signal, corresponding to extension product formed in the reaction vessel containing ddCTP, consistent with all the target nucleic acid having C in the coding strand (G in the template strand).

FIG. 6B is the electropherogram obtained for a A/A homozygote. In this instance, characteristic extension product should only be formed in the sequencing reaction containing ddATP, and this is in fact the result observed (i.e., only green extension product is formed).

FIG. 6C depicts the electropherogram obtained for a sample taken from an A/C heterozygote. As expected, the electropherogram contains two smaller peaks corresponding to signal from characteristic extension product. Labeled extension product in this instance is formed in both reaction vessels. Consequently, both blue and green signals are observed. Furthermore, the relative magnitude of the two peaks is approximately half that of the corresponding adjacent signals (i.e., the blue signal for the FAM-labeled characteristic extension product is approximately half that of adjacent blue peaks; likewise, the green signal for the JOE-labeled characteristic extension product is about half that of the signals for other JOE-labeled extension products).

### EXAMPLE 3

### Single Nucleotide Polymorphism Analysis Utilizing Single Nucleotide Methods

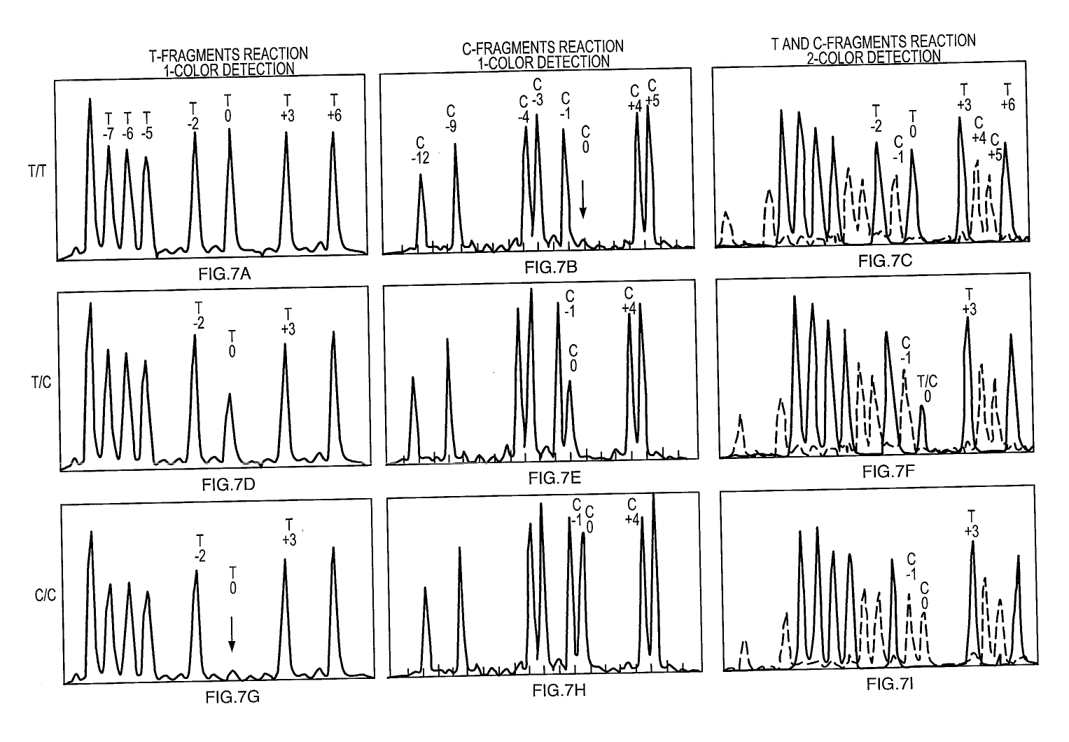

A T/C single nucleotide polymorphism was analyzed using the one nucleotide sequencing reaction approach described in Example 1. One sequencing reaction was conducted in the presence of ddTTP. Another parallel sequencing reaction was conducted in the presence of ddCTP. The extension products formed in each reaction were separately subjected to electrophoresis to generate separate electropherograms. The results are shown in the two left-most columns of electropherograms shown in FIG. 7. The peak in the electropherogram designated with an arrow and/or designated as being signal “0” is the signal corresponding to extension product in which the dideoxynucleotide was incorporated into the variant site of the extension product (i.e., the signal for characteristic extension product corresponding to the SNP site).

For comparison, two nucleotide sequencing reactions as described in Examples 1 and 2 were also conducted. In this instance, extension products from a reaction conducted with ddTTP and a separate reaction conducted with ddCTP were pooled prior to conducting electrophoresis. As described in Example 2, the primers in the two reactions were differentially labeled to distinguish the extension products. The dashed line represents extension product having ddCTP incorporated at the 3+ end; the solid line represents extension product having ddTTP incorporated at the 3′ end. Electropherograms from these set of reactions are shown in the right-most column of FIG. 7.

The first row of electropherograms in FIG. 7 (i.e., FIGS. 7A-C) is for a T/T homozygote. As expected, a signal for characteristic product is obtained in the ddTTP reaction but not the ddCTP single nucleotide reactions. The result is confirmed with the two nucleotide reaction in which the only colored product formed corresponding to the SNP site corresponds to reactions conducted with ddCTP.

The bottom row of electropherograms in FIG. 7 (i.e., FIGS. 7G-I ) is for the opposite situation in which sample is obtained from a C/C homozygote. The results would be expected to be the reverse of those obtained for the T/T homozygote. Namely, characteristic extension product would only be expected to be formed in reaction mixtures containing ddTTP. This is in fact the result observed.

Finally, the middle row of electropherograms (i.e., FIGS.7D-F), is for a T/C heterozygote. In this instance, the signal for characteristic extension product in the single nucleotide reactions is anticipated to be roughly half the magnitude of the other extension products, and this is the result observed. The electropherogram for extension products generated in the two nucleotide method shows two half-height peaks corresponding to the characteristic extension product generated in both of the sequencing reactions.

The results obtained in both Example 1 and 2 illustrate that the relative magnitude of any given peak in the electropherogram is remarkably constant for the various extension products formed within a sample (i.e., neighboring peaks have similar magnitudes) and from sample to sample. As noted above, this consistency means that the identity of a nucleotide at a variant site can be determined by conducting a partial sequencing reaction with only a single nucleotide. As these examples demonstrate, a heterozygote peak corresponding to the variant site is approximately half the relative height of a homozygote peak. Thus, in the example just described for a T/C polymorphism, if a single partial sequencing reaction is conducted with the nucleotide T, then a fall-height peak, a half-height peak and a peak having a non-detectable signal for the characteristic extension product corresponds to a T/T homozygote, a T/C heterozygote and a C/C heterozygote, respectively.

It is understood that the examples and embodiments described herein are for illustrative purposes only and that various modifications or changes in light thereof will be suggested to persons skilled in the art and are to be included within the spirit and purview of this application and scope of the appended claims. All publications, patents and patent applications cited herein are hereby incorporated by reference in their entirety for all purposes to the same extent as if each individual publication, patent or patent application were specifically and individually indicated to be so incorporated by reference.

What is claimed is:

1. A method for analyzing a variant site in a target nucleic acid, comprising:
(a) partially sequencing the target nucleic acid by conducting template-dependent primer extension reactions with the target nucleic acid serving as template in the presence of the four deoxynucleotides (dATP, dTTP, dCTP and dGTP) and one to three non-extendible nucleotides, each non-extendible nucleotide selected to be complementary to a different base potentially occupying the variant site of the target nucleic acid, whereby a plurality of extension products of differing size are formed, and wherein the extension products include in part
a first extension product if a first of the non-extendible nucleotides is present and complementary to the base occupying the variant site of the target nucleic acid,
a second extension product if a second of the non-extendible nucleotides is present and complementary to the base occupying the variant site of the target nucleic acid, and
a third extension product if a third of the non-extendible nucleotides is present and complementary to the base occupying the variant site of the target nucleic acid; and
(b) detecting the presence or absence of the first, second and/or third extension product(s) as an indication of the base occupying the variant site of the target nucleic acid.

2. The method of claim 1, wherein the sequencing step is conducted with a labeled primer, whereby the extension products are labeled and the detecting step comprises detecting the presence or absence of labeled first, second and/or third extension product.

3. The method of claim 2, further comprising comparing the magnitude of a signal from the labeled first, second and/or third extension products with the magnitude of a signal from one or more other labeled extension products.

4. The method of claim 1, wherein the sequencing step is conducted with differentially labeled non-extendible nucleotides, and the detecting step comprises detecting the presence or absence of labeled first, second and/or third extension product.

5. The method of claim 1, further comprising a labeling step in which the first, second and third extension products if present are labeled with different labels, and the detecting step comprises detecting the presence or absence of labeled first, second and/or third extension product.

6. The method of claim 1, further comprising separating the extension products by size.

7. The method of claim 6, wherein the separating step comprises separating the extension products by electrophoresis.

8. The method of claim 1, wherein the sequencing step is conducted with two or three non-extendible nucleotides and comprises dividing the target nucleic acid between different reaction vessels and conducting one of the extension reactions within each vessel in the presence of a primer and one of the non-extendible nucleotides, the primer and/or non-extendible nucleotide differing between reaction vessels.

9. The method of claim 8, wherein primers in the different reaction vessels bear different labels.

10. The method of claim 1, wherein the sequencing step is conducted with two or three differentially labeled non-extendible nucleotides in a single reaction vessel with different non-extendible nucleotides bearing different labels, and the detecting step comprises detecting the presence or absence of labeled first, second and/or third extension product.

11. A method for analyzing a variant site in a target nucleic acid, comprising:
(a) partially sequencing a target nucleic acid by conducting template-dependent extension reactions with the target nucleic acid serving as template in the presence of the four deoxynucleotides (dATP, dTTP, dCTP and dGTP) and a single nonextendible nucleotide selected to be complementary to a base potentially occupying the variant site of the template nucleic acid, whereby a plurality of extension products of differing size are formed, and wherein the extension products include in part a first extension product if the single non-extendible nucleotide is complementary to the base occupying the variant site of the target nucleic acid; and
(b) detecting the presence or absence of the first extension product as an indication of the base occupying the variant site of the target nucleic acid.

12. The method of claim 11, wherein the sequencing step is conducted with labeled primer, whereby the extension products are labeled, and the detecting step comprises detecting the presence or absence of labeled first extension product.

13. The method of claim 12, wherein the label is selected from the group consisting of a fluorophore, a chromophore, a radioisotope, an enzyme substrate, an electron dense agent, a magnetic particle and a mass label.

14. The method of claim 11, wherein the single non-extendible nucleotide is labeled such that the first extension product, if formed, is labeled, and detection comprises detecting the presence or absence of labeled first extension product.

15. The method of claim 11, further comprising labeling the plurality of extension products, and the detecting step comprising detecting the presence or absence of labeled first extension product.

16. The method of claim 11, wherein the primer comprises the M13 universal primer sequence.

17. The method of claim 1 1, wherein the 3′ end of the primer is at least 10 bases upstream from the variant site on the target nucleic acid once annealed to the target nucleic acid.

18. The method of claim 11, wherein the 3′ end of the primer is at least 25 bases upstream from the variant site on the target nucleic acid once annealed to the target nucleic acid.

19. The method of claim 11, wherein the 3′ end of the primer is at least 50 bases upstream from the variant site on the target nucleic acid once annealed to the target nucleic acid.

20. The method of claim 11, wherein the first extension product is 50 to 500 nucleotides in length.

21. The method of claim 20, wherein the first extension product is 50 to 200 nucleotides in length.

22. The method of claim 11, further comprising amplifying the target nucleic acid prior to the sequencing step.

23. The method of claim 22, wherein the amplifying step comprises amplifying the target nucleic acid under conditions whereby one or more specific primer binding sites are introduced into the amplified target nucleic acid.

24. The method of claim 11, further comprising separating the plurality of extension products according to size.

25. The method of claim 24, wherein the separating step comprises separating the extension products by electrophoresis.

26. The method of claim 11, wherein the primer is labeled such that labeled extension products are formed, and the detecting step comprises detecting the presence or absence of labeled first extension product, and further comprising comparing the magnitude of a signal for the labeled first extension product with the magnitude of a signal for one or more other labeled extension products.

27. The method of claim 26, further comprising comparing the magnitude of a signal for the labeled first extension product with an average value of the magnitudes of a plurality of signals for other labeled extension products.

28. The method of claim 26, wherein the target nucleic acid is obtained from a sample from a diploid subject and the target nucleic acid comprises a first and/or a second target nucleic acid that potentially differ in sequence at the variant site, the variant site being occupied by a first or second base, the first base being complementary to the non-extendible nucleotide, whereby
if the magnitude for the signal for the first extension product is substantially equivalent to the signal magnitude for one or more of the other extension products, then the subject is homozygous for the first base,
if the magnitude for the signal for the first extension product is substantially non-detectable, then the subject is homozygous for the second base, and
if the magnitude of the signal for the first extension product is approximately half that of the magnitude for one or more of the other extension products, then the subject is a heterozygote.

29. The method of claim 28, wherein the extension products are size-separated by electrophoresis.

30. A method for analyzing a variant site in a target nucleic acid, comprising:
(a) partially sequencing the target nucleic acid by conducting template-dependent primer extension reactions with the target nucleic acid serving as template in the presence of the four deoxynucleotides (dATP, dTTP, dCTP and dGTP) and a first and a second non-extendible nucleotide, the non-extendible nucleotides selected to be complementary with the bases potentially occupying the variant site of the target nucleic acid, whereby a plurality of extension products of differing size are generated, and wherein the extension products include in part
a first extension product if the first non-extendible nucleotide is complementary to the base occupying the variant site of the target nucleic acid, and
a second extension product if the second non-extendible nucleotide is complementary to the base occupying the variant site of the target nucleic acid; and
(b) detecting the presence or absence of the first and second extension product as an indicator of the base occupying the variant site of the target nucleic acid.

31. The method of claim 30, wherein the extension reactions are conducted in two reaction vessels, the extension reaction in the first reaction vessel comprising the template-dependent extension of a first primer in the presence of the four deoxynucleotides and the first non-extendible nucleotide and the extension reaction in the second reaction vessel comprising the template-dependent extension of a second primer in the presence of the four deoxynucleotides and the second non-extendible nucleotide, both the first and second primer hybridizing to the target nucleic acid.

32. The method of claim 31, wherein the first primer and the second primer are differentially labeled, whereby the first and second extension products, if formed, bear different labels, and the detecting step comprises detecting the presence or absence of labeled first and second extension product.

33. The method of claim 30, wherein the sequencing step is conducted in a single reaction vessel and the first and second non-extendible nucleotides are differentially labeled, whereby the first and second extension products are labeled if formed, and the detecting step comprises detecting the presence or absence of labeled first and second extension product.

34. A method for analyzing a variant site in a target nucleic acid, comprising:
(a) partially sequencing the target nucleic acid by conducting template-dependent primer extension reactions with the target nucleic acid serving as template in the presence of the four deoxynucleotides (dATP, dTTP, dCTP and dGTP) and a first, a second and a third non-extendible nucleotide, the non-extendible nucleotides selected to be complementary with the bases potentially occupying the variant site of the target nucleic acid, whereby a plurality of extension products of differing size are generated, and wherein the extension products include in part
a first extension product if the first non-extendible nucleotide is complementary to the base occupying the variant site of the target nucleic acid,
a second extension product if the second non-extendible nucleotide is complementary to the base occupying the variant site of the target nucleic acid, and
a third extension product if the third non-extendible nucleotide is complementary to the base occupying the variant site of the target nucleic acid; and
(b) detecting the presence or absence of the first, second and third extension products as an indicator of the base occupying the variant site of the target nucleic acid.

35. The method of claim 34, wherein the extension reactions are conducted in three reaction vessels, and
(a) the extension reaction in the first reaction vessel comprises the template-dependent extension of a first primer in the presence of the four deoxynucleotides and the first non-extendible nucleotide;
(b) the sequencing reaction in the second reaction vessel comprises the template-dependent extension of a second primer in the presence of the four deoxynucleotides and the second non-extendible nucleotide; and
(c) the sequencing reaction in the third reaction vessel comprises the template-dependent extension of a third primer in the presence of the four deoxynucleotides and the third non-extendible nucleotide,
each of the primers hybridizing to the target nucleic acid.

36. The method of claim 35, wherein the first, second and third primer each bear different labels, whereby the first, second and third extension products, if formed, bear different labels, and the detecting step comprises detecting the presence or absence of labeled first, second and third labeled extension product.

37. The method of claim 34, wherein the extension reactions are conducted in a single reaction vessel and the first, second and third non-extendible nucleotides are differentially labeled, whereby the first, second and third extension products bear different labels, if formed, and the detecting step comprises detecting the presence or absence of the first, second and third labeled extension product.

38. A method for analyzing a variant site in a plurality of target nucleic acids, comprising:
(a) partially sequencing a first and second target nucleic acid potentially differing in sequence at the variant site by conducting primer extension reactions with the target nucleic acids serving as templates in the presence of four deoxynucleotides (dATP, dTTP, dCTP and dGTP) and a single non-extendible nucleotide, whereby a plurality of extension products of differing size are formed, and wherein the extension products include in part
a first extension product if the single non-extendible nucleotide is complementary to the base occupying the variant site of the first target nucleic acid, and
a second extension product if the single non-extendible nucleotide is complementary to the base occupying the variant site of the second target nucleic acid; and
(b) detecting the presence or absence of the first and second extension product as an indication of the base occupying the variant site of the first and second target nucleic acids, respectively.

39. The method of claim 38, wherein the first and second target nucleic acids are from different subjects.

40. The method of claim 38, wherein
(a) the partial sequencing of the first and second target nucleic acids is conducted in a single reaction vessel and comprises contacting the first and second target nucleic acids with differentially labeled first and second primers, the first primer specifically hybridizing to the first target nucleic acid and the second primer specifically hybridizing to the second target nucleic acid, whereby the first and second extension products become differentially labeled; and
(b) the detecting step comprises detecting the presence or absence of labeled first and second extension product.

41. The method of claim 38, wherein
(a) the sequencing step comprises
(i) conducting an extension reaction with the first target nucleic acid in a first reaction vessel in the presence of the four deoxynucleotides, the non-extendible nucleotide and a first primer bearing a first label, whereby if the single non-extendible nucleotide is complementary to the base occupying the variant site of the first target nucleic acid, then labeled first extension product is formed, and
(ii) conducting an extension reaction with the second target nucleic acid in a second reaction vessel in the presence of the four deoxynucleotides, the non-extendible nucleotide and a second primer bearing a second label, whereby if the single non-extendible nucleotide is complementary to the base occupying the variant site of the second target nucleic acid, then labeled second extension product is formed; and
(b) the detecting step comprises detecting the presence or absence of the labeled first and second extension products.

42. The method of claim 41, wherein the first and second primer have the same sequence.

43. The method of claim 42, wherein the first and second labels are different and the method fulrther comprises pooling the extension products from the first and second reaction vessel and separating the extension products according to size, and the detecting step comprises detecting the separated extension products.

44. In a method for analyzing a variant site of a target nucleic acid comprising conducting chain termination sequencing reactions with four non-extendible nucleotides with the target nucleic acid serving as template whereby a characteristic extension product is generated if the base occupying the variant site of the target nucleic acid is complementary to one of the four non-extendible nucleotides, the improvement comprising conducting the chain termination sequencing reactions with only one, two or three non-extendible nucleotides, each selected to be complementary to a base potentially occupying the variant site of the target nucleic acid.
